(19) United States
(12) Patent Application Publication
(10) Pub. No.: US 2022/0207719 A1
(43) Pub. Date: Jun. 30, 2022

(54) SYSTEM, METHOD, APPARATUS, AND COMPUTER PROGRAM PRODUCT FOR ULTRASONIC CLINICAL DECISION SUPPORT

(71) Applicant: Medi-Scan LLC, North Miami Beach, FL (US)

(72) Inventor: Richard Parker, Delray Beach, FL (US)

(73) Assignee: Medi-Scan LLC, North Miami Beach, FL (US)

(21) Appl. No.: 17/456,477

(22) Filed: Nov. 24, 2021

Related U.S. Application Data
(60) Provisional application No. 63/198,041, filed on Sep. 25, 2020.

(51) Int. Cl. G06T 7/00; G06T 7/10; G06T 17/00; G06T 5/00; G06T 5/10

(52) U.S. Cl. CPC G06T 7/0012; G06T 7/10; G06T 17/00; G06T 5/002; G06T 2207/30242; G06T 2207/10132; G06T 2207/30024; G06T 2207/20104; G06T 5/10

(57) ABSTRACT

A system, method, apparatus, and computer program product for ultrasonic clinical decision support. The system allows for the utilization of a conventional ultrasonic imaging device that can be coupled to an enhanced image processing engine to provide improved visualization of a condition of underlying tissues to show tissue boundary layers in a morphological conversion of an analog ultrasound image into a pure digital image. The CDSS provides for expedited acquisition and delivery of processed ultrasound images to the attending physician in a manner that enables fast 3D imaging and fast tracking of the patient condition. The system processes and analyzes classic analog B-Mode ultrasound, converts them into digital images, identifying hypoechoic structures in the process, thus permitting an objective evaluation of tissue integrity. The CDSS provides observational metrics applied to the data to give a quantified numerical evaluation instead of a subjective guess.

### CROSS-REFERENCE TO RELATED APPLICATION

This application claims the benefit of priority of U.S. provisional application number 63/198,041 filed Sept. 25, 2020, the contents of which are herein incorporated by reference.

### BACKGROUND OF THE INVENTION

The present invention relates to clinical imaging and decision support systems and more particularly to clinical imaging and decision support systems utilizing ultrasonic imaging techniques.

Ultrasound, whether B-Mode, doppler or harmonic, is in very wide use in the medical community. However, the nature of ultrasound creates speckle noise backgrounds which interfere with interpretation.

The most popular mode of ultrasound imaging is known as “B-Mode” or Brightness Mode, whereby the intensity of the ultrasound echo is caused principally by the relatively small differences in material properties between adjacent material types, which properties are usually caused by differences of water content of the two adjacent tissue layers. However, the specific characteristics of a tissue injury can be better illustrated by the tissue structure, which is not seen in popular analog B-Mode ultrasound.

While it is known that advanced practice clinicians (APCs), particularly physician assistants (PAs) and nurse practitioners (NPs) use medical imaging for comparative purposes, most such uses employ imagery in an analog form instead of a digital form which can accurately delineate tissue structure such as the structure of fiber bundles. It should be noted that the term “digital ultrasound” is often used in a marketing sense but it is technically still an analog method.

While the prior art makes mention of the total muscle or tendon fiber bundle CSA (Cross Section Area) in studying the effects of training, there is no mention in the prior art of exploiting the digital characteristics of tendon or ligament structures by calculating the individual numbers of fiber bundles from the CSA of an individual muscle fiber bundle or tertiary tendon Fiber Bundle which permits an authoritative evaluation. Also, there is no mention in the prior art of any means of characterizing the nature of an individual tertiary fiber bundle. Patent Application Publication No.: US 2008/0108898 A1 (abandoned), entitled: Method for Producing Model of Fibrous Structure of Fibrous Tissue describes a means of identifying the outline of a tertiary fiber bundle at a particular referenced site, however, the application does not address the calculation of the CSA of a particular fiber bundle at a particular site.

Likewise, investigation into the fibrous component of muscle tissue do not employ automated analysis and counting regimens of fiber bundles, which is the determinant aspect of making the technology useful in a practical sense. Furthermore, although Mula et al. discuss an automated means of calculating the muscle Cross Sectional Area (CSA), that automated means does not calculate the number of fiber bundles enclosed in a specific CSA, which counting is essential to obtain a final authoritative analysis. Belak discloses a technique for counting glass fibers in a fiber bundle but that technique has no bearing on differentiation of living tissue fiber bundles as derived from an analog B-Mode ultrasound image.

Moreover, while ultrasonic imaging techniques have been employed for assessment of pulmonary conditions, the use of ultrasonic imaging to identify and assess pulmonary tissue structures, particularly in the COVID-19 context, are heretofore unknown.

As can be seen, there is a need for improved systems, methods, and apparatus that identifies a feature and property of a tissue injury location precisely. This allows for an appropriate therapy management, can be planned, and monitored by direct observation of the nature of the fiber bundle composition in relation to other parts of tissues which may be un-injured. There is also a need to augment the usefulness of ultrasonic imaging devices such that a true independent and objective determination can be made of the subject living tissue.

### SUMMARY OF THE INVENTION

In one aspect of the present invention, a method of assessing a fibrous tissue within a target tissue of a body via ultrasonic imaging is disclosed. The method includes acquiring, via an ultrasonic imaging device, a first ultrasonic image of the target tissue of a patient. The first ultrasonic imagine forming a cohesive set of ultrasonic image frames utilizing a specified point of care ultrasound (POCUS) protocol. The first ultrasonic image is transmitted from the ultrasonic imaging device to a computing device. A user interface of the mobile computing device provides for cropping to designate a region of interest (ROI) within the target tissue. The first ultrasonic image is transmitted via a communications network to a data repository in communication with a server. An image processing engine on the server applies a predictive analytical imagery (PAI) process to the first ultrasonic image. The PAI process employs an image segmentation process on each ultrasonic image frame in the cohesive set of ultrasonic image frames to provide a digital 2D image stack. The digital 2D image stack is assembled into an articulated 3D image stack.

In some embodiments, the method includes displaying, the articulated 3D image stack on a display as a 3D model of a tissue structure of the target tissue, the 3D model presenting the tissue structure as a series of fine lines with a background speckle noise removed.

In some embodiments, the method includes acquiring, via the ultrasonic imaging device, a second ultrasonic image of the target tissue at the ROI after a predetermined temporal period. The second ultrasonic image is transmitted to the server via the communications network. The first ultrasonic image is compared to the second ultrasonic image to determine a difference in the fibrous tissue of the target tissue.

In some embodiments, the method includes applying, via a brightness index engine, a fast Fourier transformation to the first ultrasonic image to determine a first brightness index of the first ultrasonic image. The first brightness index provides an expression of a relative presence of fibrous tissue deposits within the target tissue at a first temporal period.

In some embodiments, the method includes applying, via the brightness index engine, the fast Fourier transformation to the second ultrasonic image to determine a second brightness index of the second ultrasonic image. The second brightness index provides an expression of the relative presence of fibrous tissue deposits within the target tissue at a second temporal period. The first brightness index is compared to the second brightness index, wherein when the first brightness index is higher than the second brightness index, an extent presence of the fibrous tissue is decreasing, and when the first brightness index is lower than the second brightness index, the extent of the fibrous tissue is increasing.

In some embodiments, the method includes transmitting the first brightness index and the second brightness index to the mobile computing device. The first brightness index and the second brightness index are displayed on the mobile computing device.

In some embodiments, the method includes determining a fiber count in the first ultrasonic image, determining the fiber count in the second ultrasonic image, and displaying the fiber count over a temporal period.

In some embodiments, the target tissue is a pulmonary tissue, and the PAI determines a figure of merit for the fiber count in the pulmonary tissue based on a number of tissue boundaries identified in the ROI.

In some embodiments, the method includes determining whether a local fluid collection in the pulmonary tissue has imparts a displacement of the pulmonary tissue.

In some embodiments, the method includes comparing the displacement against an extravascular lung water (EVLW) and a blood deposit.

In some embodiments, the target tissue is a musculoskeletal (MSK) tissue. In this case, the method includes determining a region of plastic deformation in the MSK tissue based on the fiber count.

In other aspects of the invention, an apparatus for enhancing an ultrasonic image of a body tissue is disclosed. The apparatus includes a server executing computer program code to host a cloud based ultrasonic image processing service, the server having at least one processor and at least one memory. Computer program code executing on the server provides instructions to receive the ultrasonic image of a target tissue from a mobile computing device via a communications network. The ultrasonic imagine includes a cohesive set of ultrasonic image frames utilizing a specified point of care ultrasound (POCUS) protocol to capture a region of interest (ROI) in the target tissue. Computer program code executing on the server storing the ultrasonic image in a data repository. The server applies a predictive analytical imagery (PAI) process to the ultrasonic image. The PAI process employs an image segmentation on each ultrasonic image frame in the cohesive set of ultrasonic image frames to provide a digital 2D image stack. Computer program code executing on the server assembles the digital 2D image stack into an articulated 3D image stack.

In some embodiments, computer program code executing on the server provides instructions to display the articulated 3D image stack on a display as a 3D model of a tissue structure of the target tissue. The 3D model presents the tissue structure as a series of fine lines with a background speckle noise removed.

In some embodiments, the apparatus includes computer program code executing on the server to compare a first ultrasonic image to a second ultrasonic image to determine a difference in the tissue structure of the target tissue.

In some embodiments, the apparatus applies a fast Fourier transformation to the first ultrasonic image to determine a first brightness index of the first ultrasonic image. The first brightness index provides an expression of a relative extent of a fibrous tissue deposit within the target tissue at a first temporal period.

In some embodiments, the apparatus includes computer program code executing on the server applying the fast Fourier transformation to the second ultrasonic image to determine a second brightness index of the second ultrasonic image. The second brightness index provides an expression of the relative extent of the fibrous tissue deposit within the target tissue at a second temporal period. Computer program code executing on the server compares the first brightness index to the second brightness index. When the first brightness index is higher than the second brightness index, the relative extent of the fibrous tissue deposit is decreasing. When the first brightness index is lower than the second brightness index, the relative extent of fibrous tissue deposit is increasing.

In some embodiments, the apparatus includes computer program code executing on the server transmitting one or more of the first brightness index and the second brightness index to the mobile computing device via the communications network.

In some embodiments, the apparatus includes computer program code executing on the server determines a fiber count in the ultrasonic image. The server provides a display the fiber count over a temporal period.

In some embodiments, the target tissue is a pulmonary tissue and the PAI process determines a figure of merit for the pulmonary tissue based on the fiber count identified in the ROI.

In some embodiments, the apparatus includes computer program code executing on the server that determines whether a local fluid collection in the pulmonary tissue imparts a displacement of the pulmonary tissue.

In some embodiments, computer program code executing on the server compares the displacement against an extravascular lung water (EVLW) and a blood deposit.

In some embodiments, the target tissue is a musculoskeletal tissue, (MSK) tissue. In this embodiment, computer program code executing on the server determines a region of plastic deformation in the MSK tissue based on the fiber count.

In other aspects of the invention, an ultrasonic clinical decision support system (CDSS) for assessing a fibrous tissue within a target tissue of a body is disclosed. The CDSS includes an ultrasonic imaging device that is configured to capture an ultrasonic image of the target tissue of a patient. The ultrasonic image includes a cohesive set of ultrasonic image frames utilizing a specified point of care ultrasound (POCUS) protocol. A mobile computing device is configured to receive the ultrasonic image from the ultrasonic imaging device. A user interface of the mobile computing device is configured to allow a user to designate a region of interest (ROI) within the target tissue via a cropping of the ultrasonic image. The system includes a server executing computer program code to host a cloud based ultrasonic image processing service, the server having at least one processor and at least one memory. A data repository is in communication with the server. The data repository is configured to store the ultrasonic image of the target tissue. An image processing engine on the server applies a predictive analytical imagery (PAI) process to the ultrasonic image. The PAI process employs an image segmentation on each ultrasonic image frame in the cohesive set of ultrasonic image frames to provide a digital 2D image stack. Computer program code, executing on the server assembles the digital 2D image stack into an articulated 3D image stack.

In some embodiments, the CDSS includes computer program code, executing on the server displays the articulated 3D image stack on a display as a 3D model of a tissue structure of the target tissue. The 3D model presents the tissue structure as a series of fine lines with a background speckle noise removed.

In some embodiments, the CDSS includes computer program code, executing on the mobile computing device that displays the articulated 3D image stack on a display as a 3D model of a tissue structure of the target tissue. The 3D model presenting the tissue structure as a series of fine lines with a background speckle noise removed.

In some embodiments, the CDSS includes computer program code, executing on the server that compares a first ultrasonic image to a second ultrasonic image to determine a difference in the fibrous tissue of the target tissue over a temporal period.

In some embodiments, the CDSS includes a brightness index engine executing computer program code that applies a fast Fourier transformation to the ultrasonic image to determine a brightness index of the ultrasonic image. The brightness index provides an expression of a relative presence of fibrous tissue deposits within the target tissue at a specified temporal period.

In some embodiments, the CDSS includes computer program code, executing on the server compares a first brightness index of a first ultrasonic image to a second brightness index of a second ultrasonic image. When the first brightness index is higher than the second brightness index, an extent of the fibrous tissue is decreasing. When the first brightness index is lower than the second brightness index, the extent of the fibrous tissue is increasing.

In some embodiments, the CDSS includes computer program code, executing on the server to transmit the first brightness index and the second brightness index to the mobile computing device via a communications network. A display on the mobile computing device displays the first brightness index and the second brightness index on the mobile computing device.

In some embodiments, the CDSS includes computer program code, executing on the server to determine a fiber count in the first ultrasonic image. Computer program code, executing on the server determines the fiber count in the second ultrasonic image. The server transmits the fiber count in the first ultrasonic image and the second ultrasonic image to the mobile computing device via a communications network. Computer program code, executing on the mobile computing device displays the fiber count in the first ultrasonic image and the second ultrasonic image over the temporal period on the display of the mobile computing device.

In some embodiments, the target tissue is a pulmonary tissue. In this embodiment, the PAI determines a figure of merit for the fiber count in the pulmonary tissue based on a number of tissue boundaries identified in the ROI.

In some embodiments, computer program code, executing on the server determines whether a local fluid collection in the pulmonary tissue imparts a displacement of the pulmonary tissue.

In some embodiments, computer program code, executing on the server compares the displacement against an extravascular lung water (EVLW) and a blood deposit.

In some embodiments, the target tissue is a musculoskeletal (MSK) tissue. In this embodiment, computer program code, executing on the server determines a region of plastic deformation in the MSK tissue based on the fiber count.

These and other features, aspects and advantages of the present invention will become better understood with reference to the following drawings, description and claims. dr

### BRIEF DESCRIPTION OF THE DRAWINGS

FIG. 1 is a schematic diagram and process flow for the ultrasonic clinical decision support system of the present invention.

FIG. 2 is a diagram showing an initial region of interest (ROI) according to aspects of the present invention.

FIG. 3 is a diagram illustrating ultrasonic scanning techniques.

FIG. 4 is a diagram illustrating a plurality of ultrasound image layers.

FIG. 5 is an image showing an enhanced ROI selection at an initial stage.

FIG. 6 is an image showing an enhanced ROI selection at a final or subsequent stage.

FIG. 7 is a 3-dimensional rendering of processed ultrasound image layers depicting a normal lung tissue.

FIG. 8 is a 3-dimensional rendering of processed ultrasound image layers depicting a diseased lung tissue.

FIG. 9 is a chart showing a fiber bundle count, density, and area along an ROI.

FIG. 10 presents an image comparison between a previous image and a subsequent image for the ultrasonic clinical decision support system.

FIG. 11 illustrates a status chart of a brightness index over a temporal period.

FIG. 12 is a stress strain diagram for providing clinical decision support in a Musculo-skeletal application.

FIG. 13 includes images showing the transformation of ultrasound image data to a 3D model of the tissues.

FIG. 14 shows a representative comparison of patient visits over time report.

FIG. 15 is an image showing an identification of organ dimension references.

FIG. 16 is an image illustrating an image “Tag” feature in a carpal tunnel syndrome application.

FIG. 17 is an image further illustrating a carpal tunnel syndrome application.

FIG. 18 is an image showing a detection and prediction report in a musculoskeletal application.

FIG. 19 is a representative system architecture according to aspects of the invention.

FIG. 20 is an alternative representation of a system architecture, showing a client app features and server-side application features.

FIG. 21 is an operations and data flow chart for the system.

FIG. 22 is simplified process flow chart.

FIG. 23 is a flow chart showing operations of a predictive analytics module.

FIG. 24 is a general process flow for the system.

FIG. 25 includes images showing a personal fingerprint of tendons corresponding to the backscattering of the US wave at the interfascicular structures. (a) Echotexture of B-mode image and (b) zoom of backscattered signal at the interfascicular structures.

FIG. 26 illustrates images showing an implementation an image segmentation process.

FIG. 27 is an ultrasound image data of a Section of an Achilles tendon B-Mode scan.

FIG. 28 is a 3D representation of an Achilles tendon processed with predictive analytical imaging (PAI), revealing structure loss.

FIG. 29 is a system diagram illustrating various operational modes.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, embodiments of the present invention provide a system, method, apparatus, and computer program product for ultrasonic clinical decision support. The system allows for the utilization of a conventional ultrasonic imaging device that can be coupled to an enhanced image processing of the captured ultrasonic image layers or outputs of which contribute to an improved visualization of a condition of the underlying tissues of interest. The present invention concerns the elimination of ultrasound gray scale and proceeds to perform digital image segmentation analysis using digital measurements of tissue boundary layers.

Although associated comparison of medical images is frequently used in medical applications, aspects of the present invention incorporate specific techniques used in a morphological conversion of an analog ultrasound image into a pure digital image.

As seen in the simplified system diagram of FIG. 24, aspects of the present invention expedite the acquisition and delivery of ultrasound images to the attending physician in a manner that enables fast 3D imaging and fast tracking of the patient condition. The system provides a novel medical imaging technology and applications or APPs (user and server side), which feature interpretation based on observation rather than inference. An observation is something that can be easily seen whereas an inference is a guess or idea that needs to be supported by evidence.

The system processes and analyzes classic analog B-Mode ultrasound, converts these grey scale images into digital images, identifying hypoechoic structures in the process, thus permitting an objective evaluation of tissue integrity. Hypoechoic structures are typically found in tissue linings and are readily identified by our technology and are converted into fine lines with background speckle noise removed. By identifying the structure of the underlying tissue, observational metrics are applied to the data to give a quantified numerical evaluation instead of subjective guess.

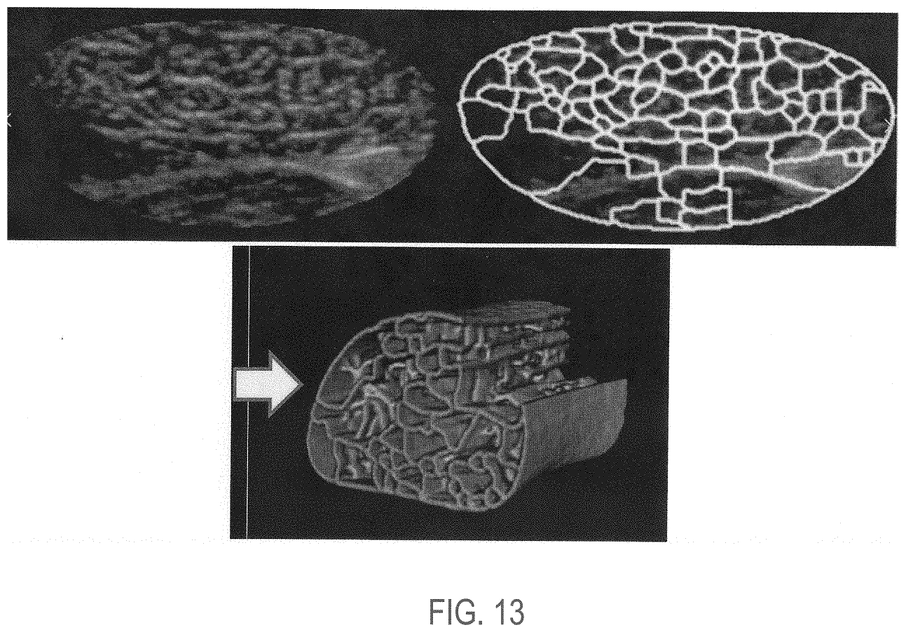

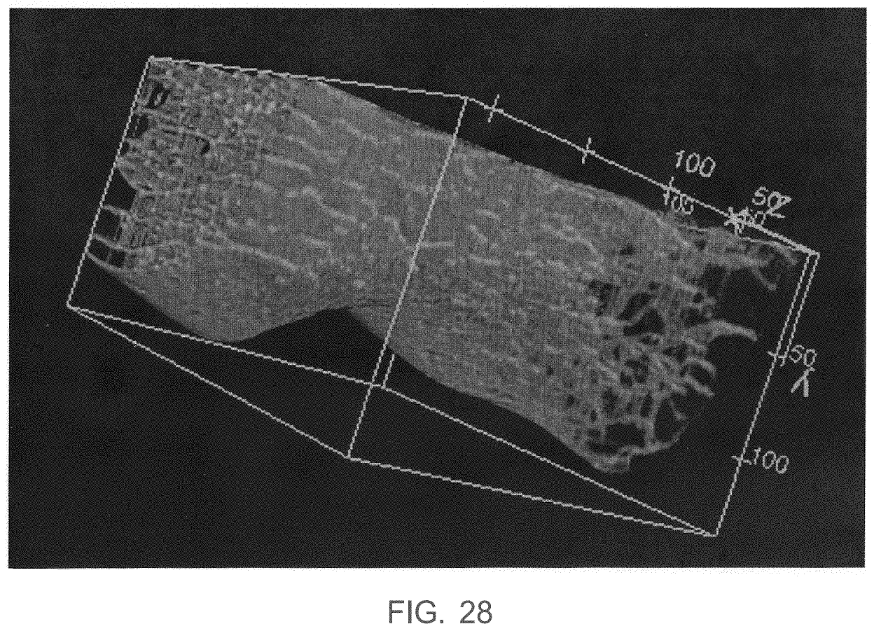

The process takes 3-5 minutes with APP “hands-on” in less than one minute. Imagery produced by the system may be seen in reference to FIG. 13, which illustrates an example of an original cropped B-Mode ultrasound compared to a final processed output of the present invention. In this case, the system is capable of reporting: “At 5.2 CM from the proximal phalanx, the number of fiber bundles decreased from a mean of 28 to 18.” For the B-Mode ultrasound on the left top, assessment of the tissue is all subjective, whereas the Medi-Scan process permits actual fiber bundle counts and is all objective. Finally, the system yields articulated 3-D images as shown in FIG. 28, which images simulate a histology examination.

Because the system may be accessed by an APP, the user only needs a cell phone or other mobile computing device to accept the images from the ultrasound imaging device. The acquired images are then uploaded to a Cloud Processor. The acquired images may be manipulated on the cell phone display and then final processed reports may be returned to the cell phone or email within 3- 5 minutes.

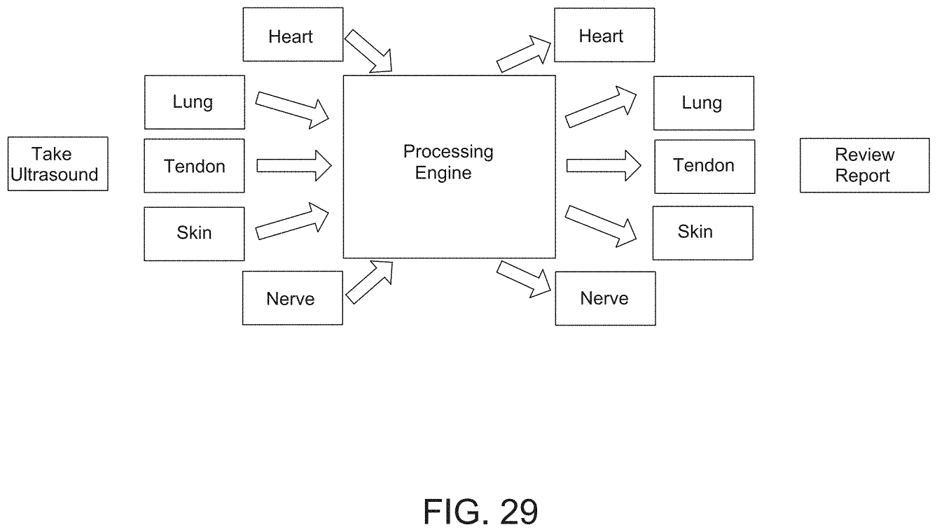

As seen in reference to FIG. 29, the present invention may be utilized for orthopedics in tracking Musculo-Skeletal (MSK) injuries, dermatology in scanning for skin lesions, chiropractic applications for monitoring nerve root inflammation, pulmonology for locating and monitoring lung inflammation as a consequence of injury or infection, and Gynecology in monitoring fetal development in 3D. In so doing, it establishes the capability of a Clinical Decision Support System (CDSS).

Image processing elements of the present invention eliminate up to 95% of the background gray scale imagery and preferentially displays water-filled boundary layers. Identification of the boundary layers can be used in illustrating the structure of dense tissue such as ligaments and tendons. Such background removal greatly assists in interpreting the region around the subject tissue suspected plasticity and enables a rapid evaluation of the subject tissue for plasticity. The present invention makes possible the use of much lower cost ultrasound devices, already utilized at a medical facility, and provides a simple description or depiction of the tissue structure. Having this property, the subject invention makes it possible to make evaluation of injured tissue a seamless process for the clinician.

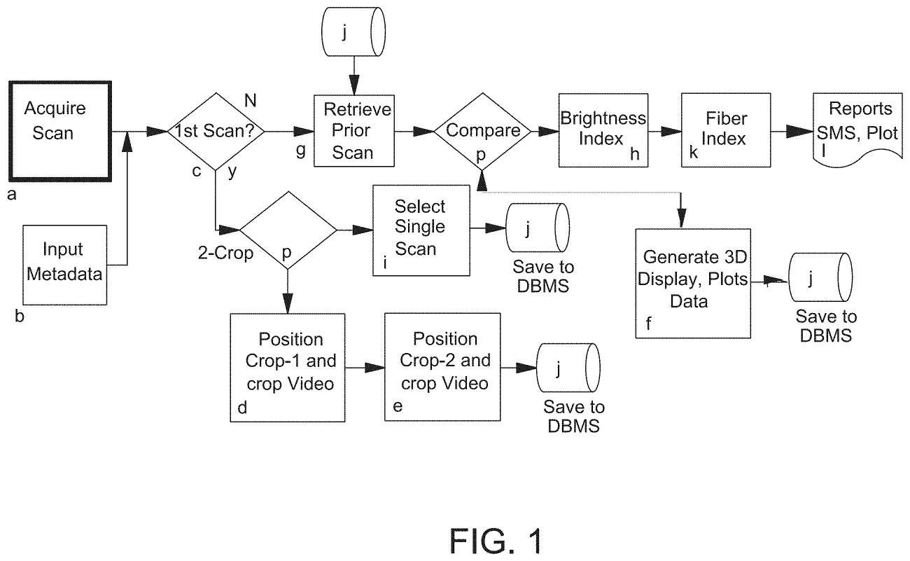

As seen in reference to FIG. 1, a Clinical Decision Support System (CDSS) includes three principal modules: An Input Module, an Image Processing Module, and a Display Module. In the Input module 2D images are acquired and a Region of Interest (ROI) is identified. In the Image Processing Module, the acquired 2D image data is transformed to identify water-filled hypoechoic structures, which are converted into fine lines with all background speckle noise removed. The Image Processing Module may also generate a 3D model of the tissue structure. The Display Module provides quantitative reports for the tissue condition as well as a progression of the tissue condition over time.

### Input Module

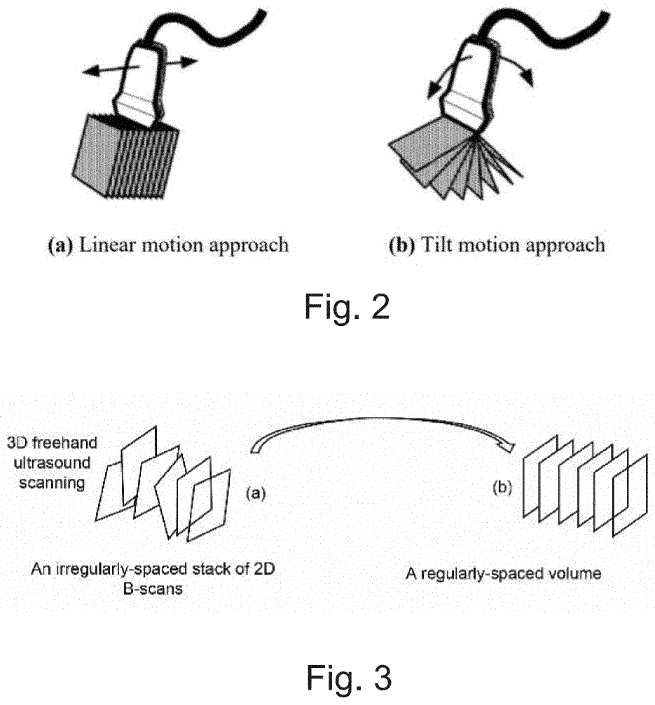

As seen in reference to FIG. 2, the system includes the acquisition of an initial scan of a patient's tissues employed in a point of care ultrasound (POCUS) protocol. The POCUS protocol utilizes a conventional ultrasonic imaging device, whether a handheld ultrasonic imaging device or a cart mounted ultrasonic imaging device. The ultrasonic imaging device is used to provide a specific guided ultrasound of the affected tissue area called a Region of Interest (ROI).

As seen in reference to FIGS. 2-4, a movement of the ultrasonic imaging device is conducted according to a specified ultrasound operator protocol to obtain one or more of a free-hand scan or a guided frame assist scan. The movement of the ultrasonic imaging device is oriented on the target tissue at the ROI such that the ultrasound image so obtained consists of a smooth series of single-frame ultrasound images.

The single-frame ultrasound images are taken in a manner to produce a single movie file from a plurality of single frame scans, all obtained using the same POCUS scanning protocol. Accordingly, the plurality of single frame scans are able to produce a substantially cohesive set of single frames. In some instances, a linear POCUS protocol may be employed, while in other instances a tilting POCUS protocol may be employed. Taken together, the plurality of image frame scans, or layers, yield a specific movie made up of single frames in a stack, as shown in FIG. 3. The POCUS protocol may be performed with a linear or a convex head on the ultrasound imaging device.

The POCUS protocol may specify one or more of the following parameters: a probe orientation, a probe frequency, a probe ROI to scan, a probe scan rate in inches per second, a probe frame rate in frames per seconds, and a probe pressure against the subject. The specific requirements of acquiring the ultrasound images are identified so that the inherent difficulty of ultrasound image evaluation is mitigated by the specified scanning protocol.

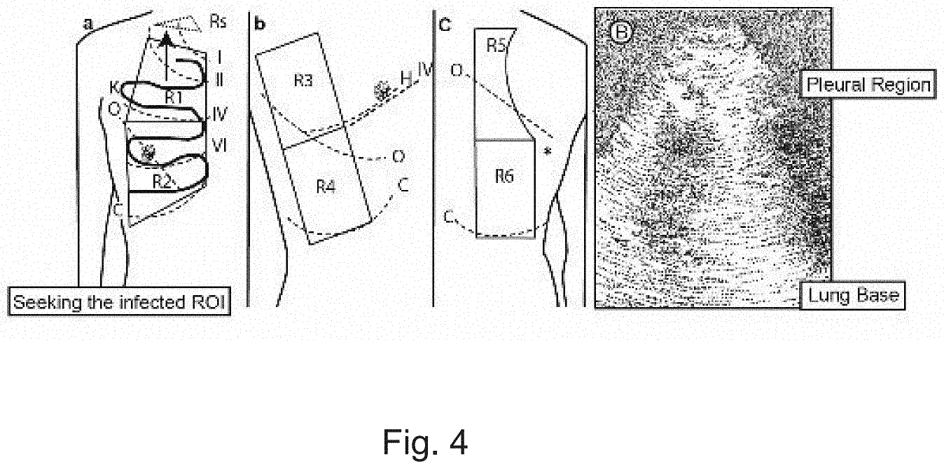

As seen in reference to FIG. 4, a pulmonary POCUS protocol shown to identify an initial ROI of a patient's lung tissue. At FIG. 4a, the lung POCUS protocol begins with an ultrasound scan of the patient's lung tissues to detect the presence of fibrous tissues within the lung, By way of non-limiting example, the lung POCUS protocol may specify initiating the scan at an inferior region of the lung R2 and scanning transversely laterally across the region R2 to a medial portion of the lung elevating the scan towards the superior region of the lung Rs and traversing back to a lateral aspect of the lung in a progressive manner until all regions of the lung tissue have been scanned.

In a preferred embodiment the use of a linear head scanner operating at a frequency of 10-12.5 MHz may be utilized. In certain situations, a convex imaging head may work well based on one or more body tissues targeted in the ROI.

The POCUS protocol is designed to take a minimum amount of time by the sonographer to capture the initial image (5-10 seconds) followed by some manual manipulation of the images, guided by an APP, in communication with the ultrasonic imaging device. The result is a sectioned group of 2D image frames arranged in a linear stack, The acquisition of said single frames merging to the file of frames making a movie is performed such that the output file so obtained may be accepted by the Image Processing Module.

Referring back to FIG. 1, the captured ultrasonic scan includes image data comprising a plurality of image layers. Metadata b may be added to the image data, reflecting one or more of a patient identification, a facility identification, an imaging technician/provider identification, or the like that are typically added to imaging systems within a clinical environment.

At block c, the image data is queried to determine whether the current image data reflects an initial scan for the subject patient and/or medical condition for which the patient is being assessed. If the image data reflects an initial scan, a determination is made whether the user wants to process a single image, or whether the user wants to process a plurality of images. If the user selects a single image layer, at block i, the user may select a desired image data from a plurality of image layers. The selected image is then saved to a database j.

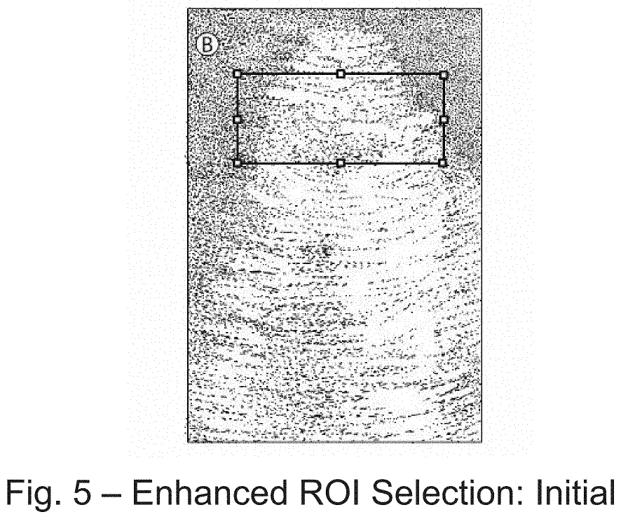

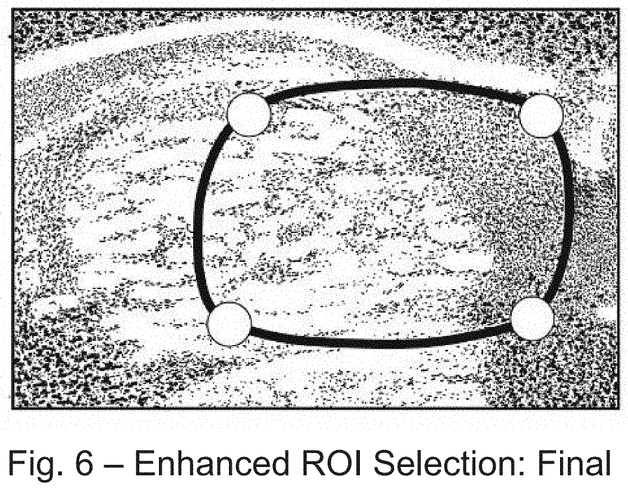

If a plurality of images is selected, at block d, the user can position and perform an initial crop of the captured image data. The initial crop, at block d, provides a general isolation of the ROI. In other embodiments a subsequent cropping operation, at block e may be employed to enable the user to make a more targeted isolation of the ROI. For example, since the acquired ultrasound image frequently has components of the image which are not of interest to the physician, the Input Module assists the physician by actively selecting a Region of Interest (ROI). In the non-limiting example of FIG. 5, a pointing device, such as a mouse, or touch screen display, may be used by the clinician to define a boundary of the ROI in the acquired image isolating the targeted ROI for subsequent image enhancement operations on the targeted ROI in the Image Processing Module.

### Image Processing Module

The image processing module includes an image processing engine f that receives the image data, which may be cropped image data, as a plurality frames arranged in the image stack. The Image Processing Engine f implements a complex math processing model employing a form of image segmentation.

In some embodiments, nonlinear hyperbolic partial differential equations are used in applying one or more shock filters, including one or more of an erosion operation and a dilation operation. The shock filters may be followed by a watershed smoothing operation and other associated processing operations. It is to be noted that there are many forms of image segmentation processes in use, and the present invention is not limited to the embodiments discussed herein.

Image Smoothing is performed using a Fast Fourier Transform which may be of the lossy and lossless form. There are four (4) types of FFT processes which are lossless. These are Run-length encoding, Entropy encoding, Huffman coding and Arithmetic encoding. These math operations all may be used to analyze the ultrasound image and filter out unwanted information.

As indicated previously, the image processing engine may be utilized for clinical applications relating to dermatology, musculoskeletal, pulmonary, nerve tissues, cardiology, and other organs. In a non-limiting embodiment, the image processing engine will be discussed in the context of musculoskeletal applications, such as equine musculoskeletal applications. These techniques and principles are applicable to other target tissues of a body.

Hyperechoic structures observed on B-Mode of normal tendons are the result of reflection and back-scattering of the US waves at the interfascicular spaces that surround fiber bundles. The interfascicular structures of equine tendon, that generate hyperechoic structures at a frequency of 7.5 MHz are those which have thicknesses larger than the acoustic wavelength λ (˜256 μm) along the axis of the US wave propagation.

A backscattering signal is a property of the internal architecture of the structure of the tissues of interest which involves a thickness, and a position relative to the US wave's propagation axis and components. Aspects of the image processing engine resolve the interfascicular structure backscatter such that a unique tissue identification may be obtained. In musculoskeletal applications, this backscatter is influenced heavily by the presence of collagen.

Biochemical analysis reveals the presence of two types of collagen: types I & III. In the normal adult horse tendon, Type I collagen predominates (95%). Collagen Type I has a low mechanical elasticity. It is highly resistant to forces applied in tension and it is mainly the fibers parallel to the transmission axis forces.

Collagen Type III occupies 3-4% of the total collagen and enters the fiber formation of small size, which is less resistant and isotropically oriented. Type III collagen is also present in the interfascicular structures that surround fibers. The molecules of both types are similar; however, the diameter of the fibrils that form the collagen type III is lower than that of those constituting the collagen of Type I. The relative proportion of these two types of collagen thus determines the mechanical properties of the tendon.

Normal healthy tendons are composed mostly of parallel arrays of collagen fibers, making up to 86% of the total dry mass closely packed together. Collagen is the main component of tendon. It represents 80% of the dry matter and 30% of the total mass. The tendon contains a large amount of water primarily bound up in the endotenon (54-85% of the total mass) and only 3% of proteoglycans, glycoproteins, and elastin. It is the wet/dry contrast of endotenon with fiber that permits highly detailed 3D images of endotenon we realize with the Morphologie technology. We further represent that this local property possesses a “fingerprint” which can be detected in the ultrasound backscatter.

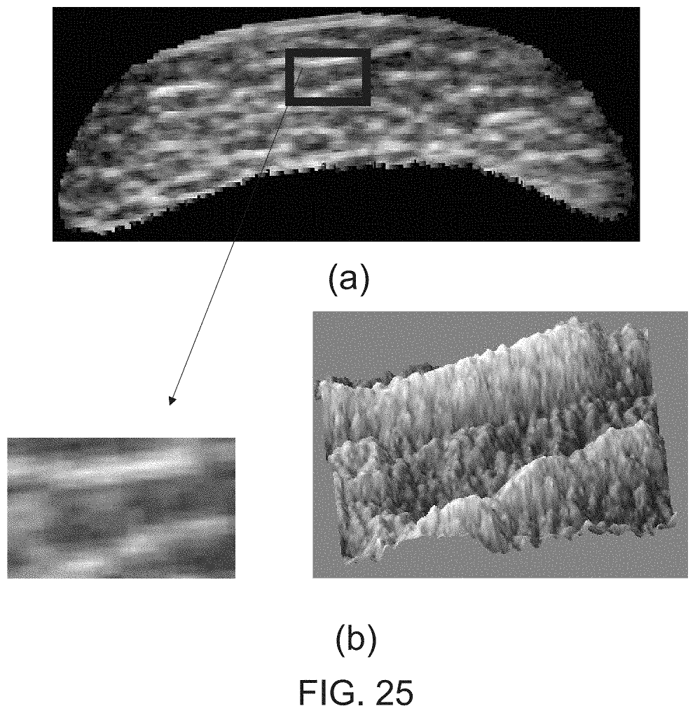

As seen in reference to FIG. 25, a personal fingerprint of tendons corresponding to the backscattering of the US wave at the interfascicular structures is shown. Sub figure (a) is an echotexture of a B-mode image and the area indicated in (b) is a magnified view of the backscattered signal at the interfascicular structures.

The image processing engine applies this property of the fingerprint, as it significantly accelerates the development of a 3D imagery of the target tissues. By employing this concept, it will be possible to tag individual elements of a structural composite such that those elements taken together completely enclose and define the structure which, in this example may be a tendon or ligament. Thus, having this definition, it now becomes possible to create individual 3D images of those unique elements which are separated from the background noise, whether it is an element or complete body part and permit a much more convenient and detailed examination of the targeted tissues.

### Correspondence between Real Structures and observed Hyperechoic Structures

Several studies were conducted to understand the internal tendon structure to document injuries and to evaluate the integrity of the equine Superficial Digital Flexor Tendon (SDFT). The most familiar methods use histological correlation in vitro, which is based on a comparison between transverse B-Mode 2D US images matched with corresponding histological sections. However, these techniques are limited because it is difficult to correctly implement our unique method and to analyze the information contained on both images.

Few studies involving image segmentation of B-mode ultrasound images exist to establish a correspondence between the B-mode image and living tissue. To date, they have proven inadequate to the task for biologic targets. However, in the process of developing new techniques, we have discovered that certain tissue characterization properties exist for the backscatter signal. The notion that backscatter may contain information related to a specific tissue type is novel and presents a new and more efficient means of evaluating ultrasound images of living tissue.

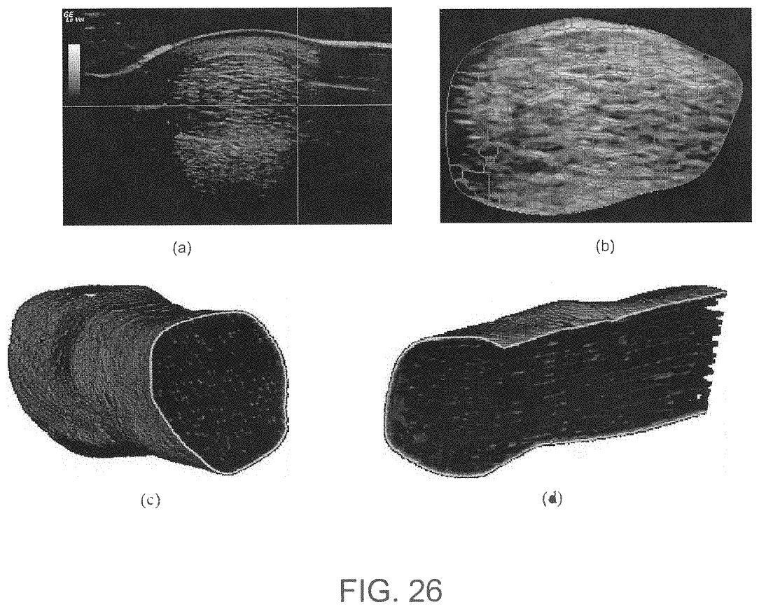

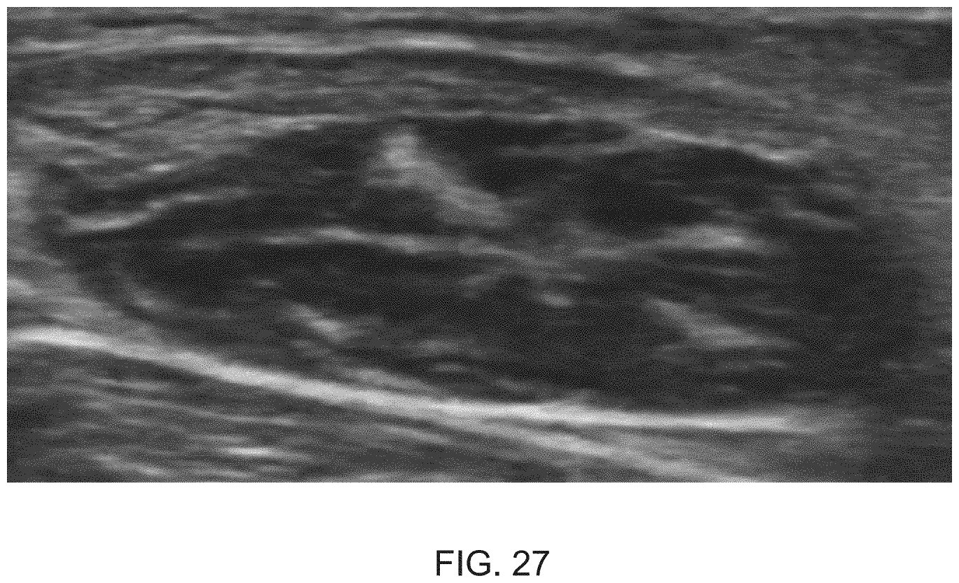

In the technology we describe as Predictive Analytical Imagery (PAI), discussed in further detail below, we apply a specialized and highly developed form of image processing to transform a blurry B-Mode ultrasound into sharp and well-defined 3D image presenting structural edges as shown in FIG. 26. Here, we see the semi-automatic interaction of cropping the normal B-Mode ultrasound of an equine tendon (FIG. 26-(a)), then Segmenting hyperechoic structures corresponding to the interfascicular structures (FIG. 26-(b)), and finally the fully processed 3D image derived from stacking successive images of tendon layers (FIG. 26-(c) and (d)).

These images are made possible by a highly refined technique derived originally using image segmentation principles, However, the techniques of the image processing engine use the principles of our PAI. In this example, we show that PAI succeeds where classic image segmentation has failed to create this type of localized accuracy for 40 years.

The PAI Image Segmentation process may be employed to obtain a “Fiber Index”. Image Segmentation algorithms used include an Edge based, a Threshold based, a Region based, a Fuzzy based, and an Artificial Neural Network based segmentation and a new segmentation-based lossless image coding (SLIC) method.

The image processing engine generates realistic 3D images of tissue structures, such as the tendon and ligament structures of FIG. 26, uses precise mathematical modeling rooted in natural physical laws and biological components of the target tissues, instead of empirical attempts to refine grayscale B-Mode ultrasound images. As a preliminary step it is necessary to locate tissue boundaries. In the musculoskeletal context, locating the interfascicular structure layers prior to such analysis utilizes the fact that these layers are highly reflective of ultrasound energy and they represent the true structure of the tissues.

The core of the PAI is derived from nonlinear hyperbolic partial differential equations (NHPDE) that are utilized for extraction of fine detail from the blurred B-Mode US image data. A brief description of a representative filter of the PAI image processing engine is written in equation (1)as follows:

$$\{ \begin{matrix}{I}_{t} + F ( {( {G}_{σ} * {I}^{0} )}_{ηη} , {( {G}_{σ} * {I}^{0} )}_{η} ) {I}_{η} = 0 in {R}^{2} \times {R}^{+} \\ I ( x , y , t = 0 ) = {I}^{0} ( x )\end{matrix} \qquad ( 1 )$$

The original B-mode image data is used as input I(x, y, t=0)=I⁰. A fingerprint function F is used to track the inter tissue structure, such as the interfascicular structures of the tendon. The fingerprint function satisfies the following:

F(0,0)=(0,0), and [(x, y)·F(x, y)]≥0

Where processes may include:
1. G_{σ}* I⁰ represents a smoothing of I⁰ using a Gaussian filter to reduce noise.
2. The detection of the tissue boundaries is guided by a gradient vector

$$η = \frac{∇ I}{‖ ∇} ‖$$

are slope in 2D plane as per detected edges.
3. Subscript η represents a derivative with respect to the gradient vector. i.e. rate of change of intensities along the slope of the gradient of that region.
4. R²×R⁺ represents a 2D positive real set.
5. The Fingerprint function F is a shock filter operator which returns ±1 for both x and y direction. The zero crossing (change in polarity) is considered as an edge. The Fingerprint function, F(x,y)=(sin(x),sin(y)) was chosen (17) to be the fingerprint function in the case of equation (1).
6. I(x, y, t=0)=I⁰(X) defines that the initial zero condition is taken from the first image data of the image data sequence (video).

A derived 2D algorithm explicit scheme may then be written as follows in equation (2):

i I_{i,j}ⁿ⁺¹=I_{i,j}ⁿ−Δt·R(I_{i,j}ⁿ) (2)

### Where:

$$\{ \begin{matrix}R ( {I}_{i , j}^{n} ) = max ( 0 , {F}_{i , j} ) {K}^{+} ( {I}_{i , j}^{n} ) + min ( 0 , {F}_{i , j} ) {K}^{-} ( {I}_{i , j}^{n} ) \\ {K}^{\pm } ( {I}_{i , j}^{n} ) = \sqrt{{( {Δ}_{x}^{\pm } {I}_{i , j}^{n} )}^{2} + {( {Δ}_{y}^{\pm } {I}_{i , j}^{n} )}^{2}} \\ {F}_{i , j} = sign {( ∇ {I}^{0 , a} )}_{i , j} \\ {Δ}_{x}^{\pm } {I}_{i , j}^{n} = \pm ( {I}_{i \pm 1 , j}^{n} - {I}_{i , j}^{n} ) , and {Δ}_{y}^{\pm } {I}_{i , j}^{n} = \pm ( {I}_{i , j \pm 1}^{n} - {I}_{i , j}^{n} )\end{matrix} \\ 1. {I}_{i , j}^{n + 1} represents {( n + 1 )}^{th} derivative of image at point i , j \\ 2. {I}_{i , j}^{n} represents {n}^{th} derivative of image at point i , j \\ 3. max ( 0 , {F}_{i , j} ) returns 1 if ∇ {I}^{0 , σ} is positive at i , j else 0 \\ 4. min ( 0 , {F}_{i , j} ) returns - 1 if ∇ {I}^{0 , σ} is negative at i , j else 0 \\ 5. {K}^{+} ( {I}_{i , j}^{n} ) is magnitude of Δ {I}_{i , j}^{n} for forward derivative \\ 6. {K}^{-} ( {I}_{i , j}^{n} ) is magnitude of Δ {I}_{i , j}^{n} for backward derivative \\ 7. {Δ}_{x}^{+} forward derivative operator along the x direction \\ 8. {Δ}_{x}^{-} backward derivative operator along the x direction \\ 9. {Δ}_{x}^{+} forward derivative operator along the y direction \\ 10. {Δ}_{x}^{-} backward derivative operator along the y direction \\ 11. sign {( ∇ {I}^{0 , σ} )}_{i , j} returns + 1 if gradient of smoothened {I}^{0} is positive at i , j . and returns - 1 if negative \qquad ( 3 )$$

Combining this algorithm with different morphological operations provides a highly accurate 3D structure.

This process may be functionally described such that the shock filter detects edges and a shock process performs dilatation (region growing) in concave signal segments and erosion (region shrinking) in convex segments. The shock filter utilizes 1^{st} and 2^{nd} derivatives of images with respect to the gradient vector. These derivatives tend to amplify noise in the image. The Gaussian smoothing filter is applied before taking the derivatives to reduce noise. The foregoing method improves the detectability of blurry edges and small structures while effectively removing speckle noise.

The image processing engine utilizes a unique fingerprint signal existing within the backscatter reflection of US waves on inter tissue structures, such as interfascicular structures, perpendicular to the propagation axis of a sufficient thickness for a given wavelength. The unique fingerprint signal exhibits properties which have yet to be fully realized using current techniques of image analysis. A useful application may be derived from the previous discovery that ultrasound tissue characterization may quantitatively differentiate infarct segments from normal myocardium in patients with remote myocardial infarction.

In former technologies all structural components are addressed as characteristics of grayscale images. As indicated, the image processing engine utilizes localized inherent properties as well which should be recognized.

Identification of a selected biologic target from the ocean of speckle and other noise in B-Mode US images presents the investigator with a unique set of challenges. For this discussion we are evaluating tendon imagery techniques associated with a specific backscatter signal. Biologic targets which exhibit wet/dry boundary layers are the most susceptible to this type of analysis. In the case of tendon, collagen plays a major role.

As an illustrative but distant example of the existence of inherent local backscattering properties, physical measurements, such as an Advanced Ultrasonic Backscatter Technique have been developed for detecting and quantifying damage from high temperature hydrogen attack (i.e., steel processing at elevated temperatures in hydrogen environments) whereas biologic measurement techniques, such as calibrated muscle backscatter (CMB), demonstrate the ability to deliver local measurements of strength and function.

Numerous parameters for measurement involving the backscatter pattern have been used such as frequency dependent backscatter, velocity ratio, spectral analysis, and spatial averaging. For bone, ultrasound backscatter parameters measured from intact proximal femurs, are significantly related (p<0.05) to structural properties and mineral density. On a microscopic level (less than or comparable to an ultrasonic wavelength), mechanical inhomogeneities inherent in tissue will scatter sound. Individual inhomogeneities are less than an ultrasound wavelength and distributed throughout the volume.

η=Backscatter coefficient=Backscatter cross section/unit volume

These Volumetric signals are very weak (20 dB down from surface reflections) but useful for imaging as they are an intrinsic property of the microstructure of tissue. Because volumetric scattering is nearly isotropic, the backscattered component is always present and is therefore representative of the tissue. This representation suggests there may be a property best described as a “fingerprint”.

From the foregoing, we can see that local inherent properties do have a role to play in suggesting a fingerprint exists, but no more. These techniques, although useful in associating backscatter with local tissue properties, have not been shown capable to deliver the fine detail needed for tissue image analysis.

The image processing engine of the present invention is not reliant on these former approaches. Instead, we take the position that a backscatter analysis yields identification of the signal through identification of a specific fingerprint. This backscattering signal fingerprint can be compared to a light spectral line of atoms and molecules, which is a technique used to identify the atomic and molecular components of stars and planets without which identification would otherwise be impossible.

The specific backscattering signal for ultrasound waves represents the signature of each target tissue structure, such as a tendon, that has larger dimensions than the wavelength and is likely perpendicular to the ultrasound wave propagation axis.

The image processing engine utilizes a compute-bound optimized processing. To generate high resolution images for new applications, each new target tissue requires a thorough investigation and analysis. We anticipate the discovery of a target tissue property may permit us to create an application unique to solving the fingerprint resolution for a specified target tissue such that the fingerprint resolution does not have to be re-computed. In addition, we then may be able to wrap that application in a shell which may be conditioned for a given target tissue. For example, consider FIG. 27 showing a transverse image of an Achilles tendon revealing scar tissue:

Now examine that same section as processed by the image processing engine, FIG. 28, which reveals endotenon structure loss typical of scar tissue. This discovery and solution will take imagery technology already developed to a new performance standard requiring far less computer processing time. The ability to extend this imagery technology to many other areas of the body and tissues may provide a straightforward application of central processing code associated with a properties shell such that the final high-definition presentation of a specific tissue type may occur in a regular and timely manner.

Thus, high-definition 3D imagery of various body tissue types from readily available 2D ultrasonic imaging devices, may expand rapidly and greatly contribute to clinical usefulness. The image processing engine, thus provides for the possibility of imaging organs internal to the abdominal cavity in such a manner that they may be virtually “removed” for inspection, rotated, and sliced to reveal specific internal structures.

As seen in the resultant image of FIG. 26 the image processing engine provides a clear display of a scar tissue area as empty (displayed as such as scar tissue is relatively dry compared to endotenon). Scar tissue is typically seen on the tendon edge for Achilles tendons. This is a prime indication of a display which may have been confused with an embolism or rupture. Such differentiation is very difficult with B-Mode even with beamforming and shearwave (both newer processing technologies). The differentiation is routine for the PAI operation, as the image processing engine can identify structural changes to obtain a count of the fiber bundles, in this instance tendons. Specific tissue differentiation will permit separation of ultrasound 3D targets for examination in the future, like histology processes.

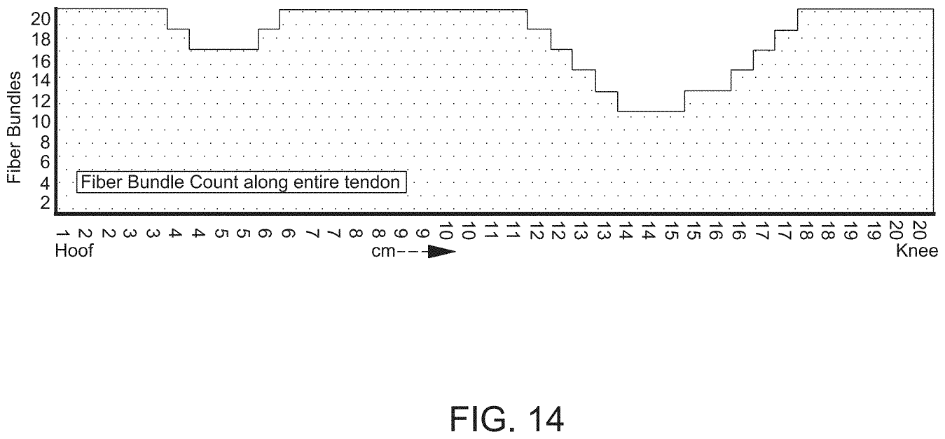

As the image processing engine permits the actual count of fiber bundles along the nominal 20-cm length of a tendon, the system is configured to display a profile of the entire tendon in one chart, such as shown in FIG. 14, thus presenting to the clinician a global at-a-glance evaluation of the overall tendon health. Subsequent ultrasounds processed with PAI can then be compared layer for layer, demonstrating whether the individual tendon layer fiber counts are increasing, which is a prime indication of improving tendon health.

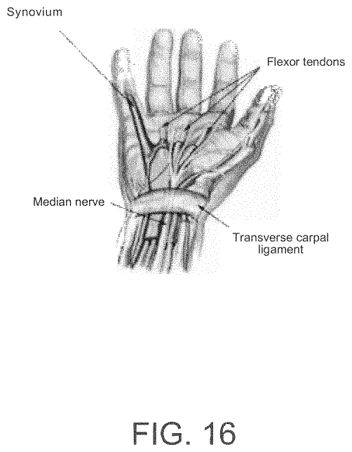

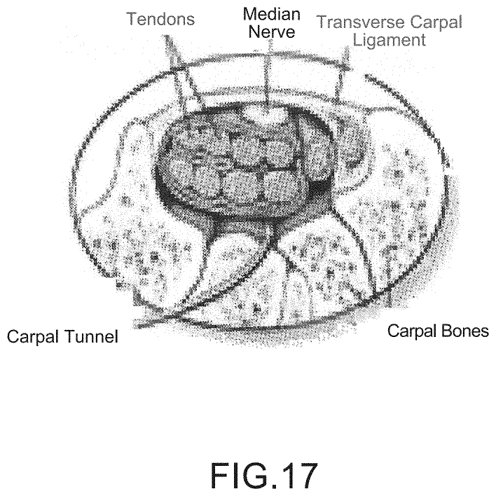

Usage of this very inexpensive technology may significantly impact the decisions and choices made concerning the use of PAI imaging. Extension of this capability to other body tissues may prove to be a useful aid in monitoring the effectiveness of a therapy for muscle spasms during administration. This capability would be very difficult with B-mode US, but routine with PAI. Furthermore, in reference to FIGS. 16 and 17 we see a clear application to carpal tunnel imagery where we should be able to tag and identify certain structures, permitting computation of tendon cross-sections and generate a “figure of merit” which may closely approximate the local pressure generated against a nerve. Such abilities provide an inexpensive, local, painless, 7-minute capability itself will give rise to routine applications which currently are unavailable in a clfinical setting.

### Display Module

The Display Module, FIG. 1 at p, formats the image data and presents the image data to the physician in a fast, convenient manner that rapidly enables the physician to form an opinion or diagnosis of the patient's condition. In some embodiments, the Display Module is configured to compare a current scan against a previous scan p, that is a previous scan and a subsequent scan.

Through the Input Module the operator may save a mark on a selected frame for the current scan experience. The Input Module may then be configured to select a corresponding frame on a subsequent scan experience which will be marked as associated with an earlier scan.

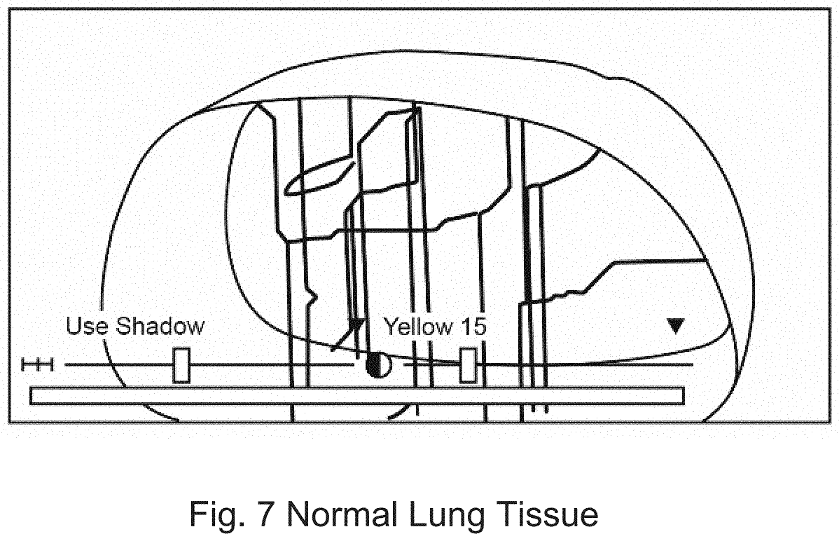

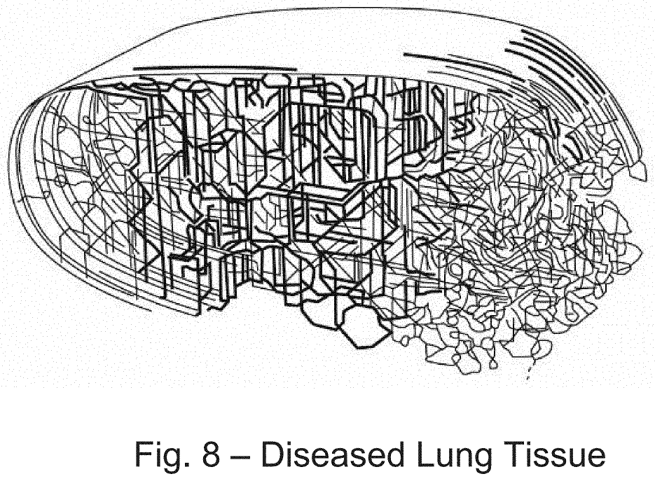

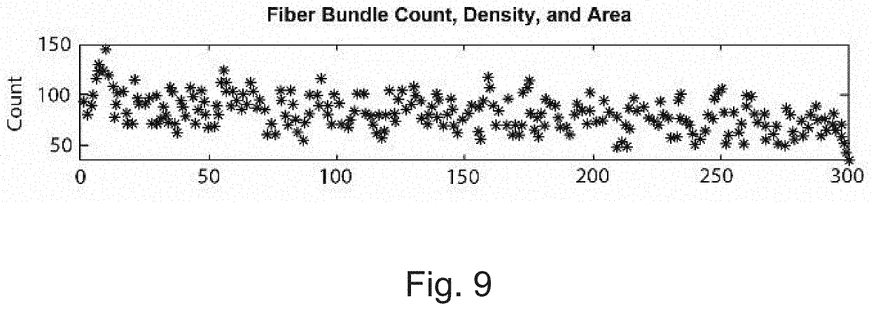

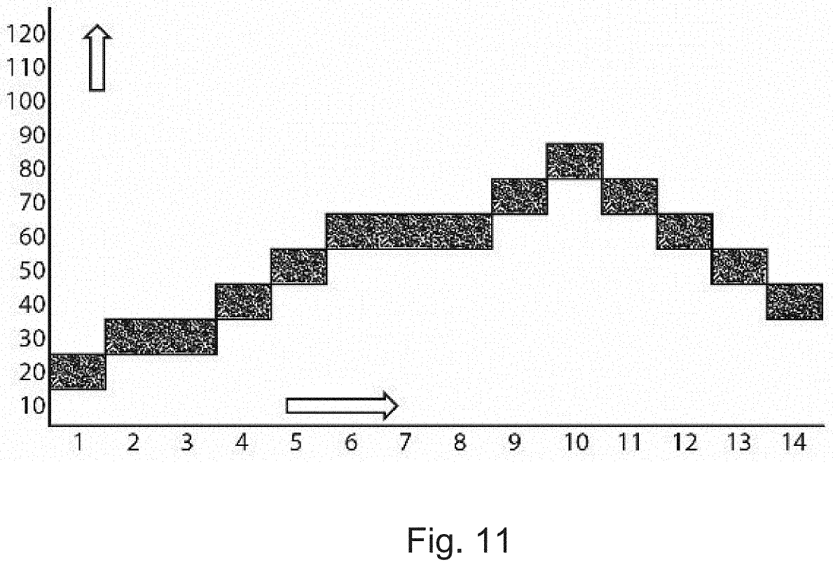

The CDSS uses the segmentation algorithm suitable for the target tissue, by way of non-limiting example, to arrive at a fibrous tissue display showing the presence and location of fibrous tissues of a lung, as shown in FIGS. 7 and 8. The analysis for creating the fibrous tissue display further lends itself to a counting method whereby a figure of merit for the Fiber Count is provided which may then be used to create a display such as is shown in FIG. 9. This fibrous display may also include a counting method whereby a figure of merit is derived for the Fiber Count, which may then be used to create a display such as is shown in FIGS. 9 and 11.

After the image processing engine processes convert the blurry analog B-Mode ultrasound into the digital 2D image stack is completed, the entire package is assembled into an articulated 3D stack, such as shown in FIG. 3. the articulated 3D stack can then be manipulated in such a manner as to enable a fast, efficient, and clear understanding of the target tissue fibrous make-up.

Once the fibrous tissue is analyzed by the segmentation program, the result may be a plot of the individual fibers for each slice from the proximal to the distal part of the slice, FIG. 3. This plot is then saved in the database j associated with the patient metadata b to preserve HIPPA requirements. The image data in the articulated 3D stack is then available for retrieval, review, and comparison against one or more of a prior or a subsequent scan.

When the CDSS receives a subsequent scan at block c, at block g, a prior scan is received from the database jto provide a comparison of the subsequent scan with the prior scan. The comparison of the respective scans enables the CDSS to provide a clinician an indication of a progression of the patient condition over a specified temporal period. The progression may be an improvement, a deterioration, or a steady state condition.

In some embodiments, a Brightness Index engine FIG. 1 h, is provided. The brightness index engine processes the ultrasound image data using algorithmic processes. These processes scan the image data and return a brightness index value to aid the diagnostic decision.

The brightness index value is an expression of the relative brightness of the lung ultrasound or an X-Ray, which is realized when ultrasound images are taken of an ROI significant scar tissue and water infiltrates. In the case of pulmonary tissues, the “Brightness Index” is a reflection of the relative brightness of an X-Ray or ultrasound image so expressed as the imaging modality is more reflective when the target pulmonary tissues includes water deposits of fibrous tissue deposits. Thus, a higher brightness index value indicates a greater presence of water deposits and fibrous tissues in the pulmonary ROI. In the case of COVID-19, this would indicate a higher infection level.

The processing time to return the brightness index value to the provider takes only minutes to facilitate the provider's diagnostic decision. A brightness index report may be provided via a plain text message or via a plot of the patient's brightness index values over a specified temporal period, such as shown in FIG. 11. The increase of the brightness index values over temporal periods 1-10 would indicate a negative progression of the patient's condition. The decrease in the brightness index values shown over temporal periods 10-14 indicate that the presence of water deposits and fibrous tissues is decreasing, indicating an improvement of the patient's condition.

The brightness Index Engine uses a Fast Fourier Transform to obtain the

“Brightness Index.” There are four (4) types of FFT processes which are lossless. These are Run-length encoding, Entropy encoding, Huffman coding and Arithmetic encoding. The choice of the FFT depends on the application and whether the process is lossy or lossless.

This final display and comparison reports is the chief contributor to gaining a more precise and consistent evaluation of the patient conditions. As each comparison report is logged, it builds up a series of instances whereby the condition of the patient can be tracked on a day-by-day basis as seen in FIG. 11.

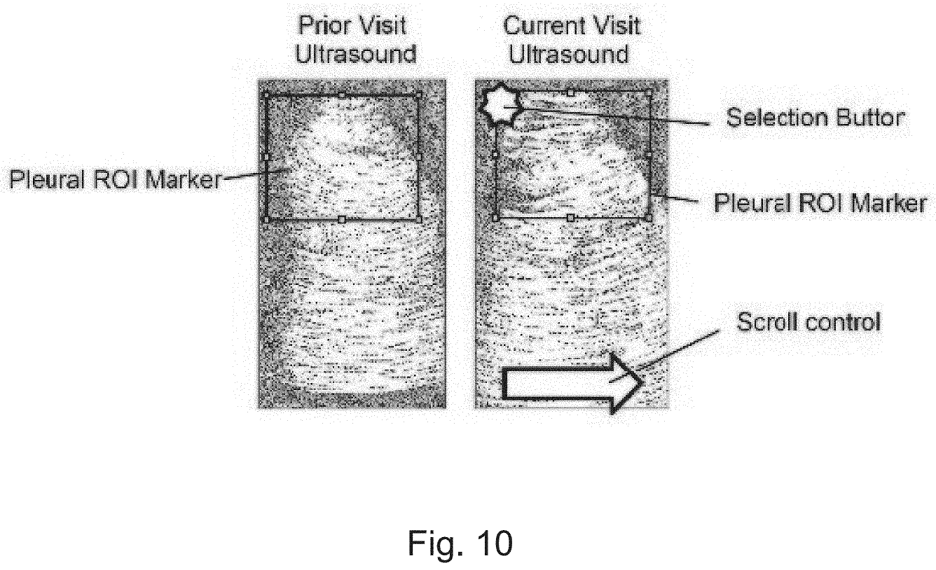

The raw B-Mode ultrasound may be presented in a placeholder featuring the current image set positioned against a prior image set such that a comparison can made between the two B-Mode images together FIG. 1p and FIG. 10. Note the image in the left in FIG. 10 represents a prior visit image, whereas the image on the right features an entire movie set of individual frames. The operation desires to find a specific frame on the right panel which matches up with the similar frame on the left panel. This is accomplished by the arrow control on the bottom of the right panel which scrolls through the movie file for the second instance until the operator spots the single frame corresponding to that on the left panel, whereby he taps the button control on the top left of the right panel of FIG. 10. As part of this specific embodiment, we present the raw B-Mode ultrasound in a placeholder featuring the current image set positioned against a prior image set such that a comparison of these two B-Mode images can be made together, FIG. 4p and FIG. 10.

The Status Chart of FIG. 11 makes it possible to issue reports in text or graphical form to a physician cell phone or tablet, informing him of the patient condition on a day-by-day basis without having to schedule a CT or an MRI scan.

### Pulmonary

Since the discovery of the COVID-19 disease, medical specialists have determined that B-Mode ultrasound can be an effective and even preferred procedure to deal with the disease, especially if the ER (Emergency Room) is complicated by a large influx of patients and compromised access to the CT scanner. In this present invention, we provide rapid evaluation of a COVID-19 patient disease state or other pneumonia concerns. This technique consists of a method and process taken together to perform an efficient and effective analysis of infected lung tissue caused by the COVID-19 virus, or any such inflammatory tissue responses associated with lung tissue, such as a pneumothorax.

The introduction of COVID-19 inserts new stressors on the healthcare system, as the disease is accompanied by features such as infections during an asymptomatic condition. Current research indicates that it is an airborne virus of serious infection rate and accompanied by a lethality ranging from 10% (elderly) to 1% (younger patients). In addition, the presence of comorbidities complicates the issue such that the infectious condition of COVID-190 is made more dangerous as the opportunistic virus seeks to exploit current and continuing weaknesses in the immune system in the face of the lack of any effective vaccine.

Ultrasound is an ideal imaging application for this condition, as it is inexpensive, fast, safe, and convenient. As previously described, ultrasound imagery is also complicated by speckle and other background noise. Consequently, conventional ultrasound images are hard to observe and interpret. To address this condition, the present invention introduces a means and mechanism to acquire the image data, prepare it for analysis, subject the image to a series of image handling and processing operations that result in a much simpler image. The resultant image data is processed to feature the underlying structural components of the target tissue in a clear and easy to understand manner, rather than the prior blurry grey scale file of the conventional ultrasound imagery.

The PAI Image Segmentation is used to determine if a local fluid collection has the effects of displacing lung tissue and compares that displacement against a finding of extravascular lung water (EVLW) and blood deposits in COVID-19 patients. This deals with two regions shown in reference to FIG. 4, the pleural cavity area where water and fibrous scar tissue accumulate and the base part of the lung which also may accumulate blood and water.

However, due to the extremis of the COVID-19 patient condition, likelihood of intubation and severity of the infection, it is difficult to take a standard CT or MRI scan and then spend an hour cleaning the machine after the scan. Through the pulmonary POCUS the clinician can use a small US device at the patient's bedside and takes an ultrasound image at a specific defined ROI.

Moreover, a major strength of this process is the ability to output comparison frames on the fly, which give unique analytical capabilities to the clinician without disturbing his workflow. For that reason, therefore, this provides a valuable part of a Clinical Decision Support System (CDSS) as defined by the FDA which enables fast, accurate and convenient clinical evaluations and diagnosis.

### 1. System and Method for Evaluating an Intubated COVID-19 Patient Disease State Based on Ultrasound Imaging

Imagery of the ROI is isolated and processed using the image segmentation tools of the image processing engine to arrive at a definitive figure of merit for the B-Lines featured in the ROI in a matter of minutes. This Figure of Merit for the fiber count is associated with a specified ROI in a manner that can be repeated through the use of a stored “Template” which takes the aforementioned ROI, saves it in a manner that it can be retrieved and placed over the current image. This can then be used as a “cropping tool” to isolate the instant ROI from the surrounding tissue for comparison to an earlier ROI taken in the same place on the same patient. This comparison will aid the physician in determining the current course of the patient.

Moreover, a Brightness Index may also be recorded and saved with the Fiber Count. This Fiber Count and Brightness Index will be a reference point for the patient condition and can be referenced against a future POCUS bedside examination to determine if the patient condition is progressing or declining.

However, even this technique (which is a major upgrade to the standard analog display) is unable to efficiently cope with the demands on ER personnel responding to a local influx of patients. The imagery data needs to be packaged and presented in such a manner that the clinician can work it into their schedule and realize the benefits of the technology, without having to push figures along and modify images. The system performs this packaging and display as part of the total algorithmic process and creates unique displays of image properties and functions for the clinician to make an informed diagnosis which is part of a CDSS.

By way of example, reports for the brightness index and the fiber count may be sent as a plain text message to the clinician's cell phone that indicates the patient's progress over time.

### 2. System and Method for Quantifying Extravascular Lung Water (EVLW)

In other aspects of the invention, the system may be utilized to determine a patient's extravascular lung water (EVLW). Imaging and indicator dilution techniques comprise the most common strategies for measuring lung water at the bedside. This medical condition is frequently associated with an accumulation of blood and water in the lung called acute respiratory distress syndrome (ARDS). Radiology remains the most accurate and most reproducible also, unfortunately, the most expensive and most difficult to implement for purposes of for routine clinical practice. The standard chest radiograph remains the best screening test for the detection of pulmonary edema. Indicator-dilution techniques are probably the best available method at present for quantitation in patient groups. However, due to the extremis of the COVID-19 patient condition, likelihood of intubation and severity of the infection, it is difficult to take a standard CT or MRI scan and then spend an hour cleaning the machine after the scan.

The present invention seeks to relieve this condition in the hospital through POCUS which uses a small probe at the bedside and takes an ultrasound image at a specific defined region. The Region of Interest (ROI), is isolated and processed using the PAI image segmentation tools to arrive at a definitive figure of merit for the B-Lines featured in the ROI in a matter of minutes. This software process is configured to accept ultrasound or X-Ray images, prepare them for analysis, and then determine if the tissue featured in each has extravascular lung water (EVLW). The Method identifies and incorporates ultrasound B-Lines supplemented with Image Segmentation to identify and quantify lung water infiltrates. B-lines allow good prediction of pulmonary congestion indicated by EVLW.

### 3. System and method for Discriminating Between Blood and Water in a COVID-19 Infected Lung Patient

This aspect of the invention uses the PAI Image Segmentation to determine if a local fluid collection has the effects of displacing lung tissue and compares that displacement against a finding of extravascular lung water (EVLW) and blood deposits in COVID-19 patients. This deals with two regions—the pleural cavity area where water and fibrous scar tissue accumulate and the base part of the lung which also may accumulate blood and water.

Although it is easy to see these deposits and understanding how debilitating it is to the patient, it is not at all easy to analyze the image, let alone quantify those findings. Our software Verification and Validation provides a unique methodology to isolate specific pleural cavity ROls and identify and quantify deep lung blood and water accumulation and perform all these analysis within 2-5 minutes.

This process is aided by the imposition of the PAI Image Segmentation model which has a pre-processor installed prior to the main processor to refine and partition the imagery This technology may also identify neutrophils, called neutrophil extracellular traps (NETs). NETs are extensive fibrous structures released extracellularly from activated neutrophils in response to infection. They are composed of cytosolic protein assembled on a scaffold of released chromatin. These structures suppress the dissemination of micro-organisms in blood by trapping them mechanically and by exploiting coagulant function to segregate them within the circulation. In addition, NET components (DNA, histone, and granule proteins) also contribute to the triggering of an inflammatory process which is detected by our system using a modified form of Image Segmentation.

With the 3D image provided by the image processing engine, the system can identify and measure tissue displacement due to the structural nature of the images.

### 4. System and Method for Tracking Progression or Decline of a COVID-19 Infected Lung Patient

This aspect of the invention uses a software technique to isolate a given Region of Interest (ROI) of lung tissue, then process that ROI using image segmentation technology in a manner to quantify the tissue featured in the ROI, then save that given ROI as a “Template” for future use on the same patient in a manner to produce an “A:B” comparison of the patient current state to a prior state.

As with previous aspects, the PAI Image Segmentation is used to determine if a local fluid collection has the effects of displacing lung tissue and compares that displacement against a finding of extravascular lung water (EVLW) and blood deposits in COVID-19 patients. This deals with two regions—the pleural cavity area where water and fibrous scar tissue accumulate and the base part of the lung which also may accumulate blood and water.

Using the POCUS, the ROI is isolated and processed using image segmentation tools to arrive at a definitive figure of merit for the B-Lines featured in the ROI in a matter of minutes. This Figure of Merit is associated with a specified ROI in a manner that can be repeated through the use of a stored “Templates” which takes the aforementioned ROI, saves it in a manner that it can be retrieved and placed over the current image, and then used as a “cropping tool” to isolate the instant ROI from the surrounding tissue for comparison to an earlier ROI taken in the same place on the same patient. This comparison will aid the physician in determining the current course of the patient

### Musculoskeletal (MSK)

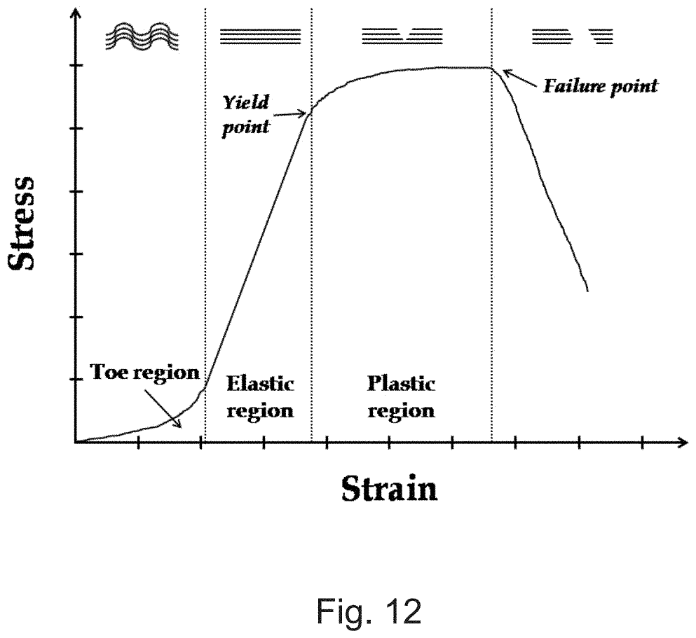

When applied to MSK conditions, the present invention provides for physician assessment of the stress/strain measurement of dense tissue (tendon and ligaments). Extended applications may be applied to soft tissue. Aspects of the present invention seek to establish an evaluation of the tendon plasticity region as separate from the elastic and failure region (FIG. 12) which will lead to a characterization of a condition of the subject tissue. Tendon plasticity, where tendon plasticity is defined as that region where the tendon fibers continue to elongate past a certain stress point but will not return to their normal condition when the stress is relieved.

In-Vivo measurements of ligaments or tendons (AKA Dense Tissue) have been extensively reported but are invasive in nature and typically require surgical intervention or device measurements. Furthermore, these measurements under consideration have been made using standard ultrasound techniques which have been shown to be characterized by poor image quality, resolution, and analog characteristics. Recently, a technique has been developed which permits measurements of the structural properties of dense tissue and reporting them in a digital format. However, these measurements have all been in the Elastic and Failure Mode of the stress/strain relationship using analog methods, whereas the total dense tissue stress response is features in three modes: Elastic, Plastic and Failure, as seen in reference to FIG. 12. The present invention provides a novel technique that uses the acoustoelastic properties of dense tissue to obtain digital characterization properties in the Plastic Region as well as the Elastic and Plastic region. This knowledge, using a noninvasive and completely safe device, shows promise of giving a complete characterization profile of dense tissue, and may have applications to soft and hard tissue as well.

Dense tissues consisting of rope-like structures, go through several discrete stages as they are the stressed, as seen in FIG. 12. Normally, a tendon is stretched and then it returns to the original length and condition. When the tendon stressed beyond a certain point, however, it enters a “Plastic Region” whereby the tendon does not return to the former shape. Beyond this, the tendon will break.

Unfortunately, this range from linear stress to plastic region to a break is very difficult to observe on B-Mode ultrasound. However, through a technique known as acoustoelasticity coupled with the PAI image processing engine, the system can identify those plastic regions and alert the sports physician.

The ability to create a complete profile of the target tissue will change the manner medicine is practiced from reporting of current conditions to a potential of predicting future responses to injury, thus making recommendation in patient behavior. For example, in practice, a ball player clobbered on the field can be evaluated at the game side, on the bench, during the game. This technology will reveal whether the black blob” shown in B mode ultrasound is a bruise or hematoma, a strain, or a break. In this case, a proper diagnosis may serve to protect the player against a career-ending injury.

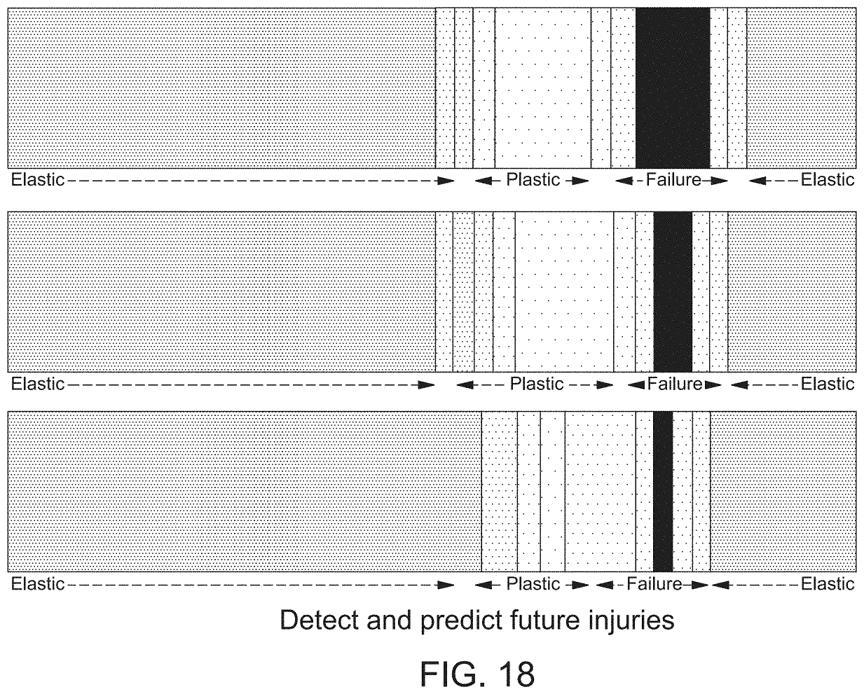

By such characterization, such as shown in reference to FIG. 18, this invention will be useful in predicting the future health of the tendon and enable the physician to plan recovery programs. The physician may compare scans taken over a recovery period, shown from top to bottom, which show an increase in the elastic region of the tendon, and a reduction in each of the plastic regions and the failure regions along the tendon, where the X-axis represents the length along the targeted tendon. In certain aspects the MSK application may allow for a prediction when an athlete can return to the game. As will be appreciated, the system may also be applied to veterinary use in equine, canine, and other animals to assess the condition and progress of the animal's injury and progression towards recovery.

In another embodiment, the present invention seeks to ascertain the plasticity of the subject tissue by means of an external apparatus which deforms the subject tissue such that the return of the tissue to the previous non-deformed state may be evaluated to determine if the subject tissue is in the state of plasticity.

The present invention makes possible the use of much lower cost ultrasound devices as the technology eliminates 95% of the background image in favor of a simple description of the structure, which makes it possible to make evaluation of injured tissue at sporting events, on the field, at the game, in real time. The system makes it possible to isolate tendon structures which have proceeded to the plastic region and thus are systematically progressing to the failure region. This identification is unique in ultrasound analysis. This identification is enabled by the identification of the subject elastic tissue through a process of tissue deformation and evaluation using the digital imaging properties of the of the image processing engine.

The system tracks fiber bundle counts, which are reduced with injury, but increased with rehabilitation. By month-to-month comparison, such as shown in FIG. 18, the system can track patient improvement digitally. This capability not seen before by physicians as the resolution perceived by the human eye of B-Mode ultrasound only appears as a blur. The PAI Image Processor resolve water-filled boundary layers which are indicative of fiber bundle outlines. Once so identified, these fiber outlines permit structural evaluations of the tendon and identify injuries expressed as broken tissue components. This is not normally possible with B- Mode ultrasound, as these images present a qualitative “black blob” to review. Because the system can quantify these outlines, it can exhibit them in a comparison manner.

Frequently, tissue structures have overlapping regions which inhibit a visual evaluation. The present system converts the underlying tissue structure to a digital form, integrate the digital form into a unified structure and then make that structure disappear or become transparent. This process we call “Tissue Tagging”, conceptually shown in reference to FIG. 16.

Once that region is identified and the tissue tag employed through a precise boundary value identification, the tagged tissue structure is made transparent in the image, so as to reveal the underlying tissue, which in the image shown are the wrist tendons and median nerve. Also, since each tendon is a separate structure, the system can compute a diameter of each tendon and so compute the internal volume of the area beneath the carpal tunnel, shown in reference to FIG. 17.

This carpal tunnel volume can then be translated directly in pressure metrics and so displayed to the patient to demonstrate that the therapy is working. Of course, the patient is usually only interested in pain relief, but in this case, we can directly display therapy effects with a digital metric and visualization. Tissue Tagging is a process formerly only available with MRI systems and very expensive software and imaging installations. It has never been seen with simple ultrasound.

### Dermatological

In another embodiment, the present invention enables a fast screening for skin cancer by highlighting certain skins regions which are of anomalous composition and comparing those regions with healthy skin surfaces which are of linear composition. Processing the boundary layer between anomalous and linear skin surfaces, the PAI can render a performance metric which will illustrate the degree of boundary value differences.

### Cardiovascular

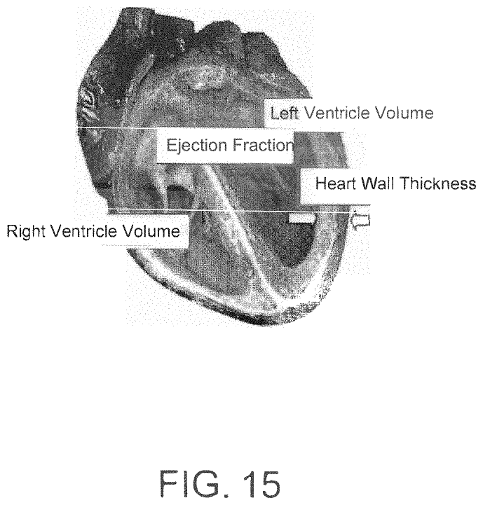

The present invention, through the use of lower frequency ultrasound, may serve up a digital 3D image of the heart which image can then be evaluated for heart disease, as the digital image can include external and internal heart measurements, shown conceptually in reference to FIG. 15.

Deep internal organ structures should be imaged with lower frequency ultrasounds, and in many cases use “harmonic” versions of the ultrasound beam. The system permits evaluation of the same tissue boundary conditions and evaluates the incident reflation (surface) as well as the backscatter reflection (interior). Once this structure is identified, the system can build a 3D articulated model which also enables internal and external measurements. For example, an external measurement may be heart wall thickness, and an internal measurement may be the left and right ventricle volume (see image above). These figures will be computed on any regular 5-minute office visit by the patient, logged and brought forward into the patient's chart for monitoring (patent in process).

### System Applications—APPs

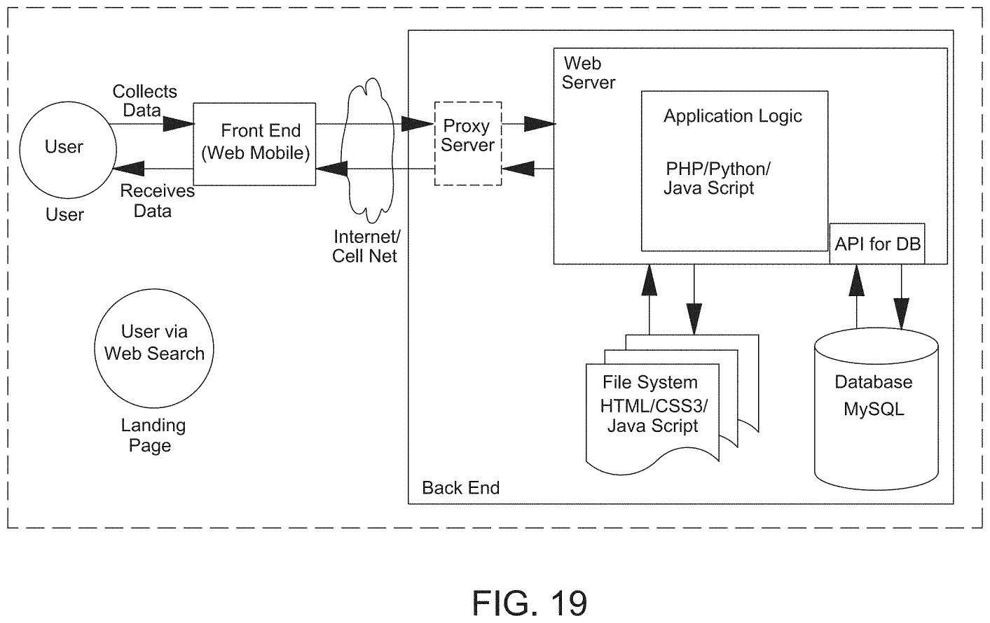

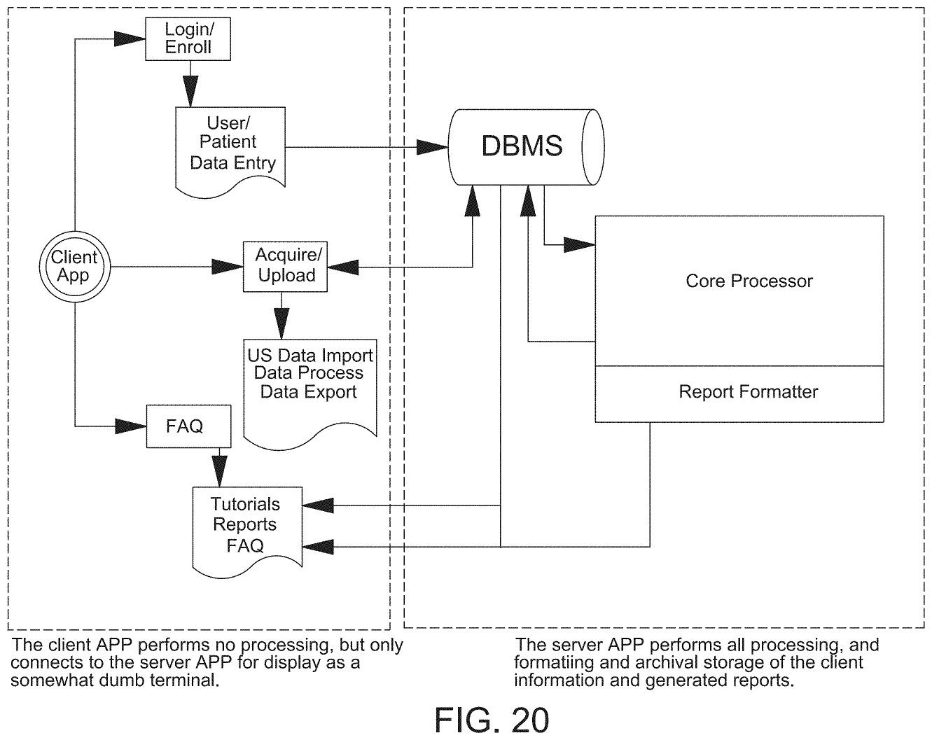

A system according to aspects of the present invention includes a mobile computing device having a display, such as a “smart” phone or tablet. The mobile computing device receives a specific ultrasound image data pattern from an ultrasound machine. An internet or a cell phone connection to a Cloud Computing or dedicated server FIGS. 19-24.

The components are enabled through an APP, a system of software processes referred to as “scaffolding” which enable the total configuration as the system and enables the various components together to be used as a productive system which will fit into the busy physicians' work-flow. Moreover, as previously described, the present invention includes a means of portraying the current state of tissue consisting of ligaments and tendons and soft tissue consisting of skin surfaces and internal body cavity organs, pulmonary, cardiac and the like.

More particularly, the present invention may encompass the use of a Cloud

Computing or a Server Computing to accept a set of layered images taken from an ultrasound machine set in a “movie mode” such that instead of a single image, the output of the ultrasound imaging device is a series of images compiled in a layered stack.

The image processing engine utilizes image segmentation, augmented using hyperbolic partial differential equations and techniques such as Predictive Analytics and Deep Learning. The image processing engine of the present invention starts with the output of the ultrasonic imaging device, which is limited. The image processing engine implements the conversion of the analog ultrasound image layers into a digital ultrasound image layer. Through a refinement processes employing artificial intelligence techniques, creates a plan view of the entire tissue area for view by the physician.

Moreover, the present invention also augments imaging by employing Deep

Learning techniques to analyze the complex imagery in such a manner that the patient history and recovery profile can be explained and set for display on a cell phone or tablet within 5 minutes, for a faster and easier form of patient education and for inclusion in the patient file.

After the modeling in the image processing engine, the input module exports the output in a series of 2D stacks of digital image layers directly corresponding to the 2D layer of analog images as obtained from the ultrasound machine. Moreover, this export is further extended and developed by the algorithmic process of the PAI.

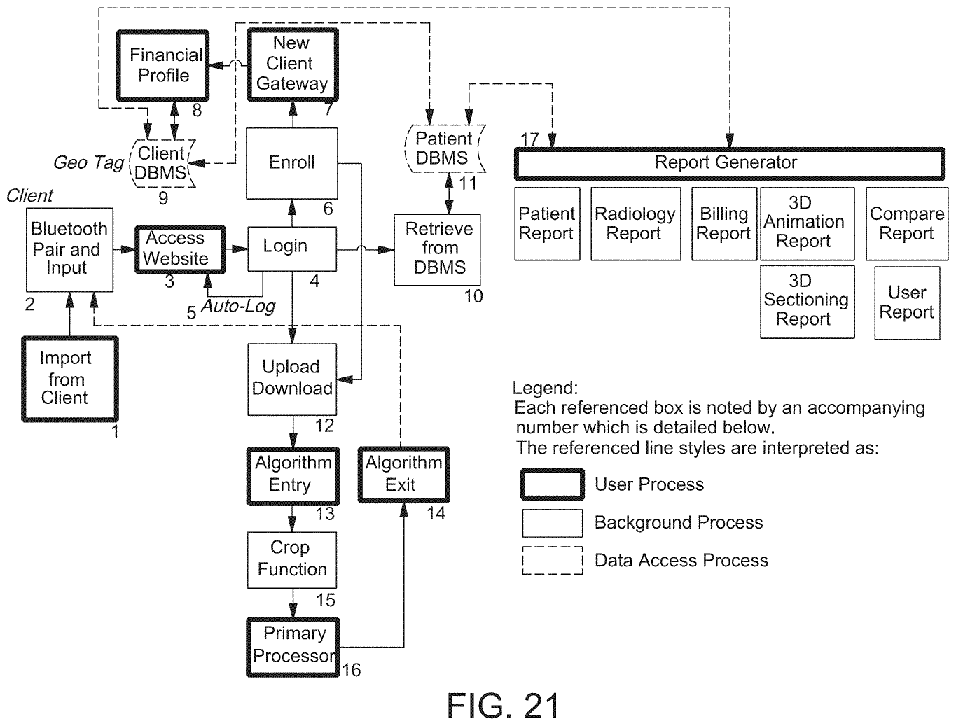

As indicated, aspects of the present invention require a significant software “scaffolding” to aggregate and process the incoming data. This scaffolding is represented in the Cloud-Server implementation as shown in FIG. 21. The “scaffolding” is described in the following description of each numbered module shown in FIG. 21.

Import from client—The customer will use his ultrasound system to capture the images, according to a defined POCUS protocol. This will result in an mp4 file that should have between 100-400 layers. It may also consist of a series of still images. Regardless, the imagery, import will either consist of a Wi-Fi, Bluetooth import or accepting a file from a USB Flash drive. If a Flash drive, the drive should be prepared first by deleting all files.

US Device Communication with Mobile Computing Device. Bluetooth pair and input—this is a typical “pairing function” which should have a “how to” or FAQ listing for instructions in the Drop-Down “Reference” button.

Access website—as this may be implemented in a thin client system, the client software only presents the user to the Company website where all communications are, by way of non-limiting example, HTMLS based. As clinician is either working with a cell phone or tablet, the tablet will have to be cell-enabled if outside a Wi-Fi network. This calls for a client-based function which prompts the user to connect properly.

Login—the system will employ an initial Enrollment and then a Login from the User. The Login will have to have provisions so that the user is identified such that patient data is restricted and adheres to HIPPA guidelines. The Login should employ a rapid identification feature such that this process is expedited.

Auto-Log—this is an expediting process that provides security but does not prove to be an irritation to the user.

Enroll—An initial sign-up Control Panel page for the program. This is described as a Control Panel as it sets system preferences and options. Including information about the provider and financial information for payments. Along with this initial information, the system may also be configured to record the GPS code of the enrollment or the GPS location for the operation. This permits attachment of a plurality of location preferences, such as shown below:

Current GPS location
GPS restriction
GPS edit
GPS range for function
Usage charge
Usage deduction with specified time
Specify no-charge Trial period
User ultrasound system preferences

New Client Gateway—this section creates a profile for the patient. The patient information may have been previously collected from the User computer system ideally (see #1), but if not, then the patient information must be collected here. The screen image for the patient data collection is shown in Phase 3—DBMS.

Financial Profile—this section may record preferences for the User and the

Patient, as far as charges are concerned. It may be preferable to embed the preferences in the enrollment process as discussed above.

Retrieve from Client DBMS—after the Login, the user can also retrieve data concerning his prior patients. This data may encompass a textual profile as well as all graphical information including processed 2D and 3D scans.

Retrieve from Patient DBMS—after the Login, the user can also retrieve data concerning patient prior visits. This data may encompass a textual profile as well as all graphical information including processed 2D and 3D scans.

Patient DBMS—the data store of the User patients. Note that this data store is linked to the Client, or User DBMS. This is discussed in Phase 3—DBMS.

Upload/Download—the system algorithm operates on an automated trigger basis. When the 2D data stack is uploaded to the Cloud Server, it is placed in a “watch folder” which automatically triggers the Core Algorithm to operate such that the Crop Function (discussed #15 below) is initiated, and then following the crop function, the algorithm processes the raw B-Mode data into the final digital data. The Download function is likewise an automated function, as the output of the Algorithm places the result in an “output folder” which triggers an automatic “push” to the client. Reports, however, are not issued until customized.

Algorithm Entry—as noted previously, the upload initiation places the raw data into a specified “watch folder” folder which involves the algorithmic processes automatically. This automatic process then initiates the Cropping Function prior to the final data processing which converts the raw ultrasound B-Mode analog data into 2D digital data and finally into 3D digital data.

Algorithm Exit—when the algorithmic process is concluded, the final file is places into an exit folder for a “push” force to the client.

Crop Function—as part of the Core Algorithm process, the system presents to the client an image of the 2D unprocessed stack. This image should be “cropped” such that the tissue of interest is separated from the background tissue of fat, muscle, cartilage, or bone. Currently, this process uses small red “dots” that are designated by the user, all outlining the tissue of interest. Other client-centric techniques such as templates and B-Splines may be developed.

PAI Primary Processor—this image processing engine designates the Core Algorithm which accepts the input data, crops the data and then solves the stack of differential equations which model the living tissue with the sample ultrasound scan, concluding in the final processed data stack output.

Report Generator—a powerful attribute of the system is the report generator which formats and presents data in a series of analytical reports. These reports will be populated with User, Patient, and graphical data as well as analytical information. The reports can be customized using drop-down menus of pre-formatted and written data populated with user and patient data. Reports may be processed for each of a Radiologist, a User, a Patient, a File, and Billing. A 3D Reveal may be provided as a presentation where the user steps through the 2/3D tissue model. A comparison of reports with reports prior visits may be used to determine progress or lack of progress.

The present invention uses existing technologies consisting of the ultrasound source device as in the ultrasound machine, the cell or internet connection to a Cloud Server and employs this total system in a manner which has never been attempted and in such a manner as to greatly broaden the scope and usage of an inexpensive and harmless high definition medical imaging device.

The present invention employs Predictive Analytics in a manner that the conversion of the set of digital layers into a final presentation which illustrates the health of the entire tendon is facilitated within a few minutes.

To facilitate patient and physician understanding, the present invention uses

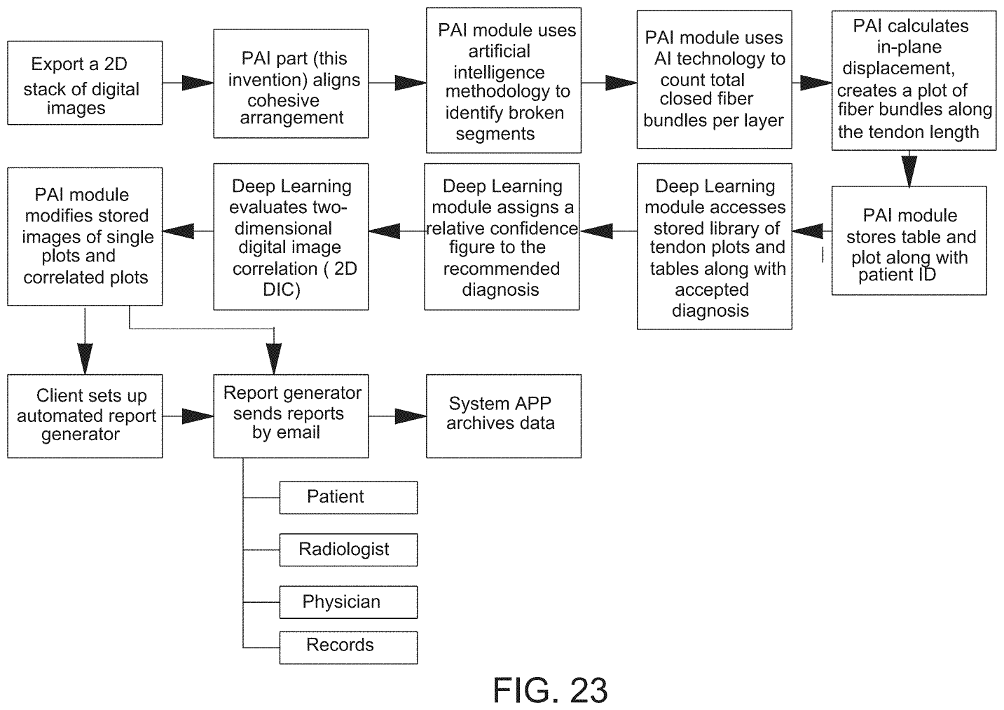

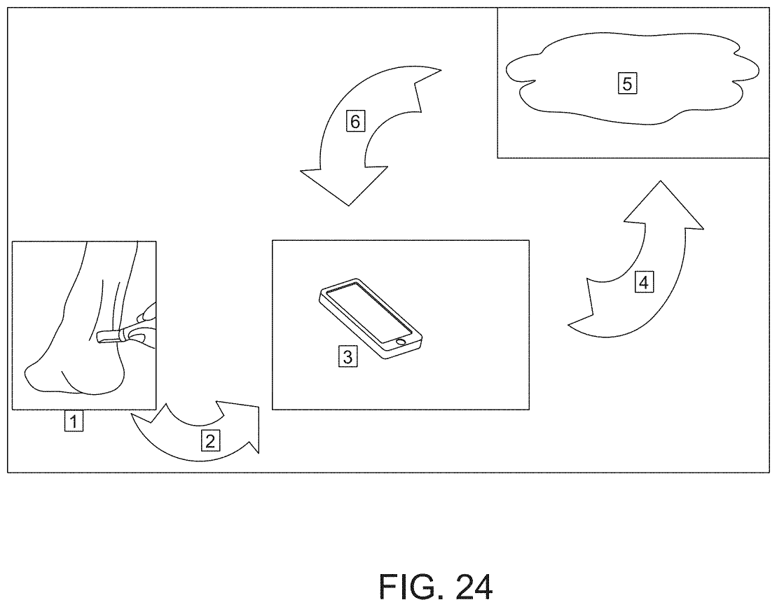

Predictive Analytical Imagery (PAI) technology, shown in FIG. 23, which yields pure digital displays. The PAI methodology is enveloped and encompassed within a total analytical, statistical display package such the final display is easy to understand and evaluated. The final display of the tissue condition contains such information as to accurately describe the targeted tissue health, thus avoiding the potential of a misdiagnosis though an incomplete output display format. Finally, in addition to the all-digital display of the targeted tissue, the output includes mapping of the processed digital output layer such the that layer compliment forms a 3D package which can then be formatted into a 3D language such as STL (Stereo Lithography) or into a proper 3D format for visualization.

The PAI receives the 2D stack of digital the ultrasonic images. The PAI aligns the frames into a cohesive arrangement. The PAI module then process the ultrasonic images via an artificial intelligence (Al) methodology to identify broken segments. The Al may then be utilized to obtain a count of a total number of closed fiber bundles per layer of the frames. The PAI may then calculate an in-plane displacement and creates a plot of fiber bundles along the tendon length. The plot is then stored along with a patient ID in a data repository. A deep learning module then accesses a the stored library of tissue plots and tables, along with an accepted diagnosis of the patient condition. The deep learning module assigns a relative confidence figure to a recommended diagnosis for a new ultrasonic image, based on the deep learning of the stored library with accepted diagnoses. The deep learning module will then evaluate a 2D digital image correlation (2DDIC). The PAI module will then modify stored images of single plots and correlated plots. A client may then set up an automated report generator. When automated reports have been specified, the reports generator sends reports via an e-mail. The e-mail reports may be sent to one or more of a patient, a radiologist, a physician, and a patient records area. The processed images are then archived in the data store.

As seen in the summary process of FIG. 24, the present invention provides a rapid, inexpensive, and safe comprehensive evaluation of the state of dense and soft tissues and display the information in a manner useful to the physician and which fits into the physician workflow. The present invention does not require expensive equipment purchases and uses the physician's own ultrasound machine and the technician's own cell phone or tablet display. Likewise, the system uses web services technology readily available to the physician over the cell or internet networks such that the physician has no demands to make on his internal computer system.

The applicator device includes an ultrasound probe connected to an ultrasound device which consists of various configurations including a local associated computer. The ultrasound probe is an applicator device which emits sound waves of various frequencies, wave shapes and amplitudes and which is directly associated with the patient by means of physical or near physical. The Client device used for data input and output may be a cell phone, tablet or computer or other such display device. The Server device may be a central computer or server farm of various configurations, which holds a central processor and storage medium. The Client is connected to the Server device by means of a network which may be a cell data network, Wi-Fi network, internet, or hard-wired network. The software is used by the Client device and the Server device and may include the following components:

(a) Client communications, which may be coded in a language specific to the type of client device being used. This permits communication between the Client and the Server.

(b) Server communications, which is coded such that it runs on the Server and facilitates communication between the Client and Server. The Server communications is presented to the network service for display by the client device, such as in HTML5, such that the final display is client-agnostic, or that it can run on multiple devices. The server communications may also include JavaScript detection Application Programming Interface (APIs) for formatting which detects the nature of the client device and thus formats the Server presentation such that the display is oriented properly, the text size is appropriate for the device, and the images are properly positioned.

(c) Specific formulaic processes, which reside on the Server and may be coded in a different computer language such that the specific algorithms of the image processing engine which define the processing of the ultrasound signal is enabled.

(d) A report formatter which uses Predictive Analytics and a Deep Learning process to collect the final processed digital images from the Core Processor and present the information in a manner which correlates the data into intercommunications protocols and web framework methodologies which govern and control communications between the Server front end display and the Server algorithmic processor.

In summary, the system may operate in the following manner. The ultrasound probe physically impressed against the tissue target which may be a ligament or tendon, or soft and hard tissue as seen in FIG. 24. In turn, the ultrasound probe emits vibrational ultrasound frequencies at a desired frequency range, such as from 3 MHz to 22 MHz These ultrasound vibrations result in internal tissue reflections which are picked up by a segmented crystal element of the ultrasound probe as incident or backscatter reflections. These reflections contain information of the structural nature of the target tissue. This information in turn is passed up to the Server by the Client and the Server algorithm software processes the information and creates a plurality of 2D images of the tissue. These images are then passed from the Ultrasound system to the Client for display to the operator.

The image analysis so obtained from the B-Mode original imagery as processed with the PAI technology will serve up a combined presentation of tissue health which would ordinarily not be available. That is, the presentation of the information contained by the analysis is facilitated by the invention technology which gathers and aggregates the information, analysis and presents the layers of processed images in a manner which can be appreciated and understood within seconds. The inventions concern not only the information data, but the Meta data associated with the data, or the fiber bundle counts. Thus, the present invention is an enablement of data processing and display which makes the entire data analysis available for use as a physician tool.

The invention will display a current condition and a current and past state of the health of the dense tissue which will be analytic of the current and past condition of the patient injury in a manner that the progress or lack thereof can be observed and understood by the patient, which will greatly aid in therapy planning and over-all case management. This information will be vital in sports medicine for equine or human applications.

The initial image may be taken using a standard B-Mode (Brightness Mode) ultrasound machine preferably employing a linear probe for surface tissue and a radial probe for deep tissue. Although B-Mode is preferred, we may use beamforming and RF-IQ technologies may be employed as well. The Client device in may be a cell phone or tablet. The Server may be a Server Farm, a Cloud Server, or an in-house server. The initial ultrasound image is taken manually using the ultrasound probe and the acoustic probe will also be used manually to take an associated image which will be directly related to the imaged body part. This ultrasound image consists of a series of 2D images obtained by placing the ultrasound machine in “movie Mode”, thus taking 2D images at a rate of 30 frames per second which is the typical frame rate of the probe head. A 10-second sweep, therefore, will produce a 300-layer stack of 2 D images, stored in what is termed an mp4 (a digital multimedia container format most commonly used to store video and audio) or a DICOM (Digital Imaging and Communications in Medicine) format—a standard for storing and transmitting medical images, each layer being approximately 0.33 mm thick. It includes a file format definition and a network communications protocol file.

This mp4 or DICOM file is then transmitted by the Client which may be a cell phone or tablet or PC by means of a hard-wired cable, Wi-Fi, or Bluetooth interface to the Server device. The Server device thereupon, uses a set of Front End processes, which may be encoded in PHP, Python and JavaScript, to present the 2D analog stack to the Core Algorithm which then proceeds to process the 2D analog stack into a 3D digital image stack. This conversion of the 2D analog stack into the 3D digital image is useful in gaining a digital representation of the ultrasound image, the resulting images are of some use to the physician. However, the conformal mapping of the digital images into a presentation useful to the physician and the Predictive Analytics enables the presentation into a set of fiber bundle counts which are directly useful in gauging the health of the affected subject tissue. The present invention accepts the final 2D digital stack of images and arranges them in a manner which permits a systematic plot of tendon fiber bundles and then, using this systematic plot, arranges said plot into mp4 or DICOM format and creates a display which can be appreciated by the physician for final display in HTMLS format. As the Client is directly connected to the website which has created the HTMLS display, the Client APP will receive and display the final image reporting format.

The Server also contains a front-end software package which is written in an appropriate programming language such as PHP, JavaScript and Python for web services inter and intra communications suitable to the Client technology. Regardless of the technology the Client APP will be able to receive and display an HTMLS image readily. In addition, there are special interfaces of the web server front end language which are devised to permit efficient handling of transmitting data to the core application. The core application may be written in a powerful multithreading language (a technique by which a single set of code can be used by several processors at various stages of execution) suitable for scientific and mathematical analysis.

In operation, the web server front end picks up the incoming mp4 or DICOM image set by means of a “push” from the Client, enabling image handing, cropping and set-up. The image set is then passed on to the PAI image processing engine receives the data package, which incorporates the special provisions of the present invention such that the final digital layer stack produced is manipulated, aggregated, analyzed and codified for display according to the previsions of the present invention which is hereby described as Predictive Analytics.

The System APP then communicates directly with the Client in a manner that permits the Client physician to specify the type of processing desired as there are several report formats from which the physician can choose. After this brief interaction, the PAI proceeds to process the image and final reports and hand off the results to the web server front end software for a “push” back from the Server APP to the Client. The physician has the use of several types of reports. As previously indicated, the final display and reporting functions can take place within 5 minutes thereby fitting within the physicians' workflow.

The Server system accesses the data consisting of client data and facility data together with the data processed by the Core Algorithm and the results as devised by the PAI algorithm and then inserts those images into pre-formatted reports such that they are available as a finished work-product, thus saving hours of office procedure time. These reports are formatted for display on the client device and are also formatted together with data accepted from the database which also contains the images.

The formatted reports may then be sent via email to their respective destinations which may include the radiologist, patient, billing clerk, file, or physician's record files. This final step enables the technology to compete the task of becoming a useful tool, instead of a curiosity. The PAI may be written and modeled in MatLab, or other suitable program, which then exports to a multithreading computer language that permits running several instances of a problem at once. However, the PAI has primitive human interface characteristics; hence it is isolated from the user by the web server front end code which is written in a very user friendly presentation language, such as PHP (PHP: Hypertext Preprocessor) and JavaScript. These tasks are wrapped up in a defined interface and module via an API (Applications Program Interface) and passed on to the web server front end for display and manipulation by the user.

The PAI processor is a unique application of predictive analytics, such that in the musculoskeletal context, the entire tendon complement of layers is examined and studied for anomalies from the mean and from the extreme. Predictive analytics is the practice of extracting information from existing data sets to determine patterns and predict future outcomes and trends. It does not predict but only forecasts what might happen in the future with an acceptable level of reliability and includes what-if scenarios and risk assessments. The PAI is applied to the final all-digital representation of the ultrasound image in a manner that permits automated evaluation of the subject images.

Once the anomalies have been determined and the extremes isolated, then a comprehensive profile for the tendon in a healthy state can be determined. Against this comprehensive profile, a layer by layer examination of the individual fiber bundle CSA is performed to determine if the CSA variants are sufficient to indicate the state of the tendon, whether it is healthy or consists of broken fiber bundles. Finally, a suitable graphical display of the Predictive Analytics is produced such that the user may readily understand the nature of the anomalies and thus gain a better appreciation of the future injury using a variant of the automated report generator.

The use and design of the present embodiment may be altered in many different manners such as placing the ultrasound and acoustoelastic probe on a mechanical arm for a motorized guidance and building in additional intelligence into the Client system so less demands are made of the Server system.

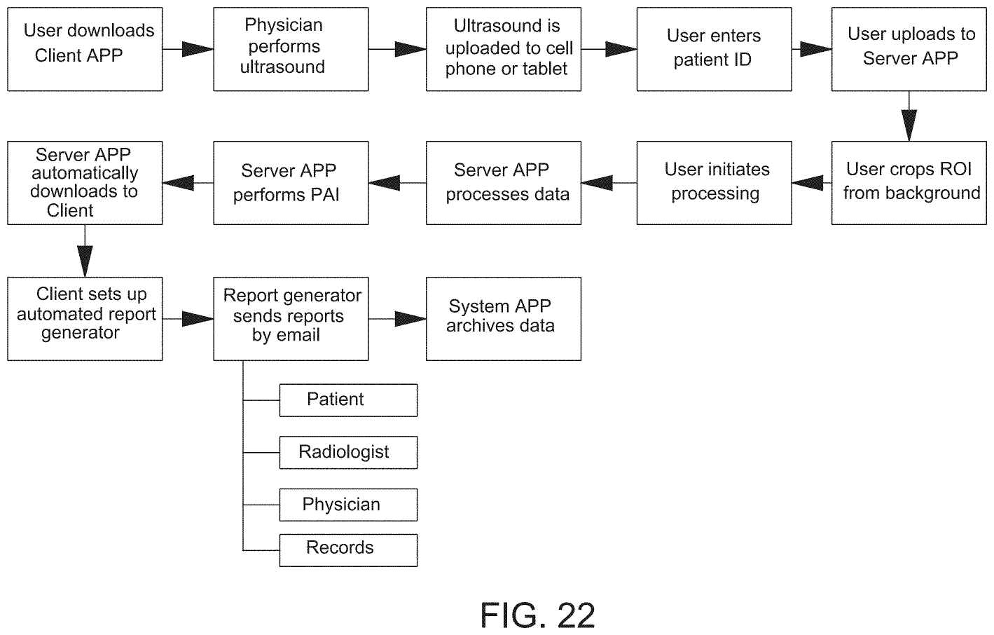

More particularly, the flow chart and data flow diagram of FIG. 22 reveals actions which take place with a given system process:

1. To initiate the process, the user downloads the Client APP from our website or from the APP store.

2. Once the client has the Client APP installed on his cell phone or tablet, he can initiate the system process with the physician taking an ultrasound scan using the tutorial information stored on the Client APP so that all ultrasound scans are taken in a similar manner.

3. The physician uploads the ultrasound to the Client display using a

Bluetooth, Wi-Fi, connection, so it is wireless and generally automatic.

4. The user now enters the patient ID, unless it has already been input.

5. The user uploads the source B-Mode ultrasound to the Server APP.

6. The user now engages a connection to the website which is enabled through the Client APP. In preferred embodiments, the website interface is in HTMLS for ease of operation. The user now crops the Area of Interest (AO1) from the background using the server programming which is engaged and controlled from the Client APP.

7. Once the crop function is completed, the user initiates the PAI process.

8. Once the Core Algorithm process is completed, it then passes the information on to the PAI process, which performs the layer identification, correlation, examination, counting, characterization and tagging for the final display formatting.

9. Once the formatting is complete the Server APP automatically downloads the data to the Client.

10. The client at this point selects the type or types of reports he wishes to see and which reports he wishes to format a prepared report for submittal by email to the radiologist, file, physician, patient or billing office.

11. While this is going on, the Server APP archives all data and the complete transaction record.

The Predictive Analytics Module (PAI) as previously described is illustrated in FIG. 23:

1. The Input Module converts analog ultrasound images to digital form in a series of stacked layers.

2. The PAI process aligns the stacked layers.

3. The PAI uses Artificial Intelligence (AI) techniques to evaluate each fiber bundle to ascertain if broken and to determine if the scan is performed properly. If not, the scan is rejected with some helpful comments. The AI is necessary as an injury will result in broken fiber bundles as will an improper placement of the ultrasound probe.

4. The PAI counts the number of fiber bundles per layer and micro-adjusts each layer for alignment to the previous layer and the succor layer.

5. The PAI creates a list of fiber bundles per layer and from that list creates a table. Finally, from this table, the PAI creates a plot.

6. The table and plot are stored along with the patient data and meta data.

7. The PAI module accesses prior patient plots and diagnosis for a correlation to ascertain if the subsequent plot is like that previously performed and with an accepted diagnosis.

8. The Deep Learning sub-module assigns a figure of merit to the subject plot.

9. The Deep Learning sub-module takes a second look at the 2D image correlation factor and makes any final alignments if necessary.

10. The PAI module stores the final 2D layer stack as a 3D coherent module

11. The client selects a desired output format or plot from the information at hand and issues commands to release or email the selected final report.

12. The report generator sends out the final formatted report to selected recipients.

13. The system may archive all data and tag all data with a metadata tag.

The system of the present invention may include at least one computer with a user interface. The computer may include any computer including, but not limited to, a desktop, laptop, and smart device, such as, a tablet and smart phone. The computer includes a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet, extranet, intranet, host server, internet cloud and the like.

The computer-based data processing system and method described above is for purposes of example only and may be implemented in any type of computer system or programming or processing environment, or in a computer program, alone or in conjunction with hardware. The present invention may also be implemented in software stored on a non-transitory computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as described in the following claims.

What is claimed is:

1. A method of assessing a fibrous tissue within a target tissue of a body via ultrasonic imaging, comprising:
acquiring, via an ultrasonic imaging device, a first ultrasonic image of the target tissue of a patient, the first ultrasonic imagine comprising a cohesive set of ultrasonic image frames utilizing a specified point of care ultrasound (POCUS) protocol;
transmitting the first ultrasonic image from the ultrasonic imaging device to a mobile computing device;
cropping the first ultrasonic image, via a user interface of the mobile computing device, to designate a region of interest (ROI) within the target tissue;
transmitting, via a communications network, the first ultrasonic image to a data repository in communication with a server, applying, via an image processing engine on the server, a predictive analytical imagery (PAI) process to the first ultrasonic image, the PAI process employing an image segmentation on each ultrasonic image frame in the cohesive set of ultrasonic image frames to provide a digital 2D image stack; and
assembling the digital 2D image stack into an articulated 3D image stack.

2. The method of claim 1, further comprising:
displaying, the articulated 3D image stack on a display as a 3D model of a tissue structure of the target tissue, the 3D model presenting the tissue structure as a series of fine lines with a background speckle noise removed.

3. The method of claim 1, further comprising:
acquiring, via the ultrasonic imaging device, a second ultrasonic image of the target tissue at the ROI after a predetermined temporal period;
transmitting, via the communications network, the second ultrasonic image to the server; and
comparing, the first ultrasonic image to the second ultrasonic image to determine a difference in the fibrous tissue of the target tissue.

4. The method of claim 3, further comprising:
applying, via a brightness index engine, a fast Fourier transformation to the first ultrasonic image to determine a first brightness index of the first ultrasonic image, the first brightness index providing an expression of a relative presence of fibrous tissue deposits within the target tissue at a first temporal period.

5. The method of claim 4, further comprising:
applying, via the brightness index engine, the fast Fourier transformation to the second ultrasonic image to determine a second brightness index of the second ultrasonic image, the second brightness index providing an expression of the relative presence of fibrous tissue deposits within the target tissue at a second temporal period; and
comparing the first brightness index to the second brightness index, wherein when the first brightness index is higher than the second brightness index, an extent presence of the fibrous tissue is decreasing, and when the first brightness index is lower than the second brightness index, the relative presence of the fibrous tissue is increasing.

6. The method of claim 5, further comprising:
transmitting the first brightness index and the second brightness index to the mobile computing device; and
displaying the first brightness index and the second brightness index on the mobile computing device.

7. The method of claim 3, further comprising:
determining a fiber count in the first ultrasonic image;
determining the fiber count in the second ultrasonic image; and
displaying the fiber count over a temporal period.

8. The method of claim 7, wherein the target tissue is a pulmonary tissue, the method further comprising:
determining, by the PAI, a figure of merit for the fiber count in the pulmonary tissue based on a number of tissue boundaries identified in the ROI.

9. The method of claim 8, further comprising:
determining whether a local fluid collection in the pulmonary tissue has imparts a displacement of the pulmonary tissue.

10. The method of claim 9, further comprising:
comparing the displacement against an extravascular lung water (EVLW) and a blood deposit.

11. The method of claim 7, wherein the target tissue is a musculoskeletal (MSK) tissue, the method further comprising:
determining a region of plastic deformation in the MSK tissue based on the fiber count.

12. An apparatus for enhancing an ultrasonic image of a body tissue, the apparatus comprising:
a server executing computer program code to host an ultrasonic image processing service, the server having at least one processor and at least one memory;
computer program code executing on the server providing instructions to receive an ultrasonic image of a target tissue from a mobile computing device via a communications network, the ultrasonic imagine comprising a cohesive set of ultrasonic image frames utilizing a specified point of care ultrasound (POCUS) protocol to capture a region of interest (ROI) in the target tissue;
computer program code executing on the server storing the ultrasonic image in a data repository;
computer program code executing on the server applying a predictive analytical imagery (PAI) process to the ultrasonic image, the PAI process employing an image segmentation on each ultrasonic image frame in the cohesive set of ultrasonic image frames to provide a digital 2D image stack; and
computer program code executing on the server assembling the digital 2D image stack into an articulated 3D image stack.

13. The apparatus of claim 12, further comprising:
computer program code executing on the server providing instructions to display the articulated 3D image stack on a display as a 3D model of a tissue structure of the target tissue, the 3D model presenting the tissue structure as a series of fine lines with a background speckle noise removed.

14. The apparatus of claim 13, further comprising:
computer program code executing on the server comparing a first ultrasonic image to a second ultrasonic image to determine a difference in the tissue structure of the target tissue.

15. The apparatus of claim 14, further comprising:
computer program code executing on the server applying a fast Fourier transformation to the first ultrasonic image to determine a first brightness index of the first ultrasonic image, the first brightness index providing an expression of a relative extent of a fibrous tissue deposit within the target tissue at a first temporal period.

16. The apparatus of claim 15, further comprising:
computer program code executing on the server applying, the fast Fourier transformation to the second ultrasonic image to determine a second brightness index of the second ultrasonic image, the second brightness index providing an expression of the relative extent of the fibrous tissue deposit within the target tissue at a second temporal period; and
computer program code executing on the server comparing the first brightness index to the second brightness index, wherein when the first brightness index is higher than the second brightness index, the relative extent of the fibrous tissue deposit is decreasing, and when the first brightness index is lower than the second brightness index, the relative extent of fibrous tissue deposit is increasing.

17. The apparatus of claim 16, further comprising:
computer program code executing on the server transmitting one or more of the first brightness index and the second brightness index to the mobile computing device via the communications network.

18. The apparatus of claim 12, further comprising:
computer program code executing on the server determining a fiber count in the ultrasonic image; and
computer program code executing on the server providing a display the fiber count over a temporal period.

19. The apparatus of claim 18, wherein the target tissue is a pulmonary tissue, and
the PAI process determines a figure of merit for the pulmonary tissue based on the fiber count identified in the ROI.

20. The apparatus of claim 19, further comprising:
computer program code executing on the server determines whether a local fluid collection in the pulmonary tissue imparts a displacement of the pulmonary tissue.

21. The apparatus of claim 20, further comprising:
computer program code executing on the server comparing the displacement against an extravascular lung water (EVLW) and a blood deposit.

22. The apparatus of claim 18, wherein the target tissue is a musculoskeletal tissue, (MSK) tissue, the apparatus further comprising:
computer program code executing on the server determines a region of plastic deformation in the MSK tissue based on the fiber count.

23. A clinical decision support system (CDSS) for assessing a fibrous tissue within a target tissue of a body, comprising:
an ultrasonic imaging device configured to capture an ultrasonic image of the target tissue of a patient, the ultrasonic imagine comprising a cohesive set of ultrasonic image frames utilizing a specified point of care ultrasound (POCUS) protocol;
a mobile computing device configured to receive the ultrasonic image from the ultrasonic imaging device;
a user interface of the mobile computing device, configured to allow a user to designate a region of interest (ROI) within the target tissue via a cropping of the ultrasonic image;
a server executing computer program code to host a cloud based ultrasonic image processing service, the server having at least one processor and at least one memory;
a data repository in communication with the server, the data repository configured to receive the ultrasonic image of the target tissue;
an image processing engine on the server, the image processing engine applying a predictive analytical imagery (PAI) process to the ultrasonic image, the PAI process employing an image segmentation on each ultrasonic image frame in the cohesive set of ultrasonic image frames to provide a digital 2D image stack; and
computer program code, executing on the server assembling the digital 2D image stack into an articulated 3D image stack.

24. The CDSS of claim 23, further comprising:
computer program code, executing on the server displaying, the articulated 3D image stack on a display as a 3D model of a tissue structure of the target tissue, the 3D model presenting the tissue structure as a series of fine lines with a background speckle noise removed.

25. The CDSS of claim 23, further comprising:
computer program code, executing on the mobile computing device displays the articulated 3D image stack on a display as a 3D model of a tissue structure of the target tissue, the 3D model presenting the tissue structure as a series of fine lines with a background speckle noise removed.

26. The CDSS of claim 23, further comprising:
computer program code, executing on the server compares a first ultrasonic image to a second ultrasonic image to determine a difference in the fibrous tissue of the target tissue over a temporal period.

27. The CDSS of claim 23, further comprising:
a brightness index engine executing computer program code applies a fast Fourier transformation to the ultrasonic image to determine a brightness index of the ultrasonic image, the brightness index providing an expression of a relative presence of fibrous tissue deposits within the target tissue at a specified temporal period.

28. The CDSS of claim 27, further comprising
computer program code, executing on the server compares a first brightness index of a first ultrasonic image to a second brightness index of a second ultrasonic image, wherein when the first brightness index is higher than the second brightness index, an extent presence of the fibrous tissue is decreasing, and when the first brightness index is lower than the second brightness index, the relative presence of the fibrous tissue is increasing.

29. The CDSS of claim 28, further comprising:
computer program code, executing on the server transmits the first brightness index and the second brightness index to the mobile computing device via a communications network; and
a display on the mobile computing device displays the first brightness index and the second brightness index on the mobile computing device.

30. The CDSS of claim 26, further comprising:
computer program code, executing on the server determines a fiber count in the first ultrasonic image;
computer program code, executing on the server determines the fiber count in the second ultrasonic image;
computer program code, executing on the server transmits the fiber count in the first ultrasonic image and the second ultrasonic image to the mobile computing device via a communications network; and
computer program code, executing on the mobile computing device displays the fiber count in the first ultrasonic image and the second ultrasonic image on the display of the mobile computing device over the temporal period.

31. The CDSS of claim 30, wherein the target tissue is a pulmonary tissue, and
the PAI determines a figure of merit for the fiber count in the pulmonary tissue based on a number of tissue boundaries identified in the ROI.

32. The CDSS of claim 31, further comprising:
computer program code, executing on the server determines whether a local fluid collection in the pulmonary tissue imparts a displacement of the pulmonary tissue.

33. The CDSS of claim 32, further comprising:
computer program code, executing on the server compares the displacement against an extravascular lung water (EVLW) and a blood deposit.

34. The CDSS of claim 30, wherein the target tissue is a musculoskeletal (MSK) tissue, the system further comprising: computer program code, executing on the server determines a region of plastic deformation in the MSK tissue based on the fiber count.
